(19) United States
(12) Patent Application Publication
(10) Pub. No.: US 2003/0187319 A1
(43) Pub. Date: Oct. 2, 2003

(54) Sentinel lymph node detecting apparatus, and method thereof

(75) Inventors: Mamoru Kaneko, Hanno-shi (JP); Toshihiro Shizuka, Tokyo (JP); Tadahiro Nakano, Tokyo (JP); Masaru Sudo, Tokyo (JP); Katsuichi Imaizumi, Tokyo (JP); Hitoshi Ueno, Tokyo (JP)

Correspondence Address: SCULLY SCOTT MURPHY & PRESSER, PC, 400 GARDEN CITY PLAZA, GARDEN CITY, NY, 11530

(73) Assignee: OLYMPUS OPTICAL CO., LTD., Tokyo (JP)

(21) Appl. No.: 10/396,040

(22) Filed: Mar. 25, 2003

(30) Foreign Application Priority Data
Mar. 29, 2002 (JP) 2002-097417
Mar. 29, 2002 (JP) 2002-097420

(51) Int. Cl. A61N 2/00

(52) U.S. Cl. 600/9

(57) ABSTRACT

A sentinel lymph node detecting apparatus comprises an endoscope, a visible-light CCU and infrared CCU, which are detachably connected to the endoscope, a superimposing circuit for superimposing the image output from the infrared CCU on the image output from the visible-light CCU, a monitor for displaying the image superimposed by the superimposing circuit, and a fluctuating magnetic field generating device for generating a fluctuating magnetic field for vibrating ferrofluid which has been accumulated in sentinel lymph nodes as a tracer beforehand, so as to heat the ferrofluid. A computer color-enhanced infrared image obtained by an infrared sensor is superimposed on an endoscope image obtained by visible-light CCD, and the synthesized endoscope infrared image is displayed on a display screen of the monitor.

This application claims benefit of Japanese Application Nos. 2002-97417 filed in Japan on Mar. 29, 2002, 2002-97420 filed in Japan on Mar. 29, 2002, the contents of which are incorporated by this reference.

### BACKGROUND OF THE INVENTION

1. Field of the Invention

The present invention relates to a sentinel lymph node detecting apparatus for detecting sentinel lymph nodes, which are lymph nodes that tumor cells entering from the primary origin of the tumor to lymphatic vessels first reach, and a detecting method thereof.

2. Description of the Related Art

In recent years, with regard to cancer in the early stages, the detection percentage thereof has been improved, and surgical removal has been widely performed. Generally, surgery for cancer in the early stages is performed with complete eradication as the object, and in many cases, multiple lymph nodes around affected portions to which cancer might have spread are removed by excision. Moreover, with surgery for cancer in the early stages, the removed lymph nodes are subjected to pathology examination following the surgery so as to confirm presence or absence of metastasis of cancer to lymph nodes, and subsequent treatment strategy is accordingly determined.

In the surgery stage, presence or absence of metastasis to lymph nodes is unknown. Therefore, in surgery for cancer in the early stages, multiple lymph nodes which are situated near affected portions are removed by excision, leading to great burden being placed on a patient. On the other hand, with breast cancer in the early stages, for example, the probability of metastasis to lymph nodes is approximately 20%. This means that with surgery for cancer in the early stages, unnecessary removal of lymph nodes has been performed for the 80% of the patients wherein metastasis has not actually occurred.

In recent years, realizing both of QOL (quality of life) of a patient and complete recovery by surgical removal for cancer has been desired. As a technique for solving this problem, sentinel node navigation surgery for preventing unnecessary removal of lymph nodes has received much attention. Description with regard to the sentinel lymph node navigation surgery will be made below in brief.

Recent researches have made clear that in the event that cancer spreads to lymph nodes, the cancer does not spread at random, but rather spreads to lymph nodes via lymphatic vessels following a certain pattern. In the event that cancer spreads to lymph nodes, it is considered that the cancer always spreads to sentinel lymph nodes. Now, the sentinel lymph node is a lymph node which cancer cells entering lymph nodes from the primary origin of cancer first reach.

Accordingly, in surgery of cancer in the early stages, judgment can be made whether or not metastasis to lymph nodes occurs, by detecting sentinel lymph nodes during surgical removal for cancer, performing biopsy, and performing speedy pathology examination. In the event that the cancer has not spread to sentinel lymph nodes, excessive excision of lymph nodes can be avoided in cancer surgery in the early stages. Conversely, in the event that the cancer has spread to sentinel lymph nodes, multiple lymph nodes near the affected portion is subjected to surgical removal according to the metastasis state in surgery of cancer in the early stages.

Excessive surgical removal of lymph nodes can be avoided for a patient whose cancer has not spread to lymph nodes, in surgery of cancer in the early stages by performing the sentinel node navigation surgery, and thus the load placed on the patient is reduced. Moreover, the sentinel node navigation surgery is not restricted to breast cancer, but rather is applied to laparotomy for a digestive organ, or the like, surgery using a peritoneoscope, or the like.

With regard to the sentinel node navigation surgery, a detecting apparatus and a detecting method for easily and accurately detecting sentinel lymph nodes, have been desired.

As the sentinel lymph node detecting method, an arrangement disclosed in Japanese Unexamined Patent Application Publication No. 2001-299676, for example, has been proposed.

With the sentinel lymph node detecting method disclosed in Japanese Unexamined Patent Application Publication No. 2001-299676, indocyanine green which is an infrared fluorescent dye is injected around a tumor as a tracer. With the sentinel lymph node detecting method, following a predetermined time period, laparotomy is performed, and near-infrared excitation rays are cast on the portion to be examined. The indocyanine green is accumulated in sentinel lymph nodes, and accordingly near-infrared fluorescence is emitted from the sentinel lymph nodes. With the sentinel lymph node detecting method, sentinel lymph nodes can be detected by converting the near-infrared fluorescence into visible light so as to observe as a visible-light image.

However, with the sentinel lymph node detecting method disclosed in Japanese Unexamined Patent Application Publication No. 2001-299676, the position of a sentinel lymph node can be identified only up to a depth of several millimeters from the surface. Therefore, with the sentinel lymph node detecting method disclosed in Japanese Unexamined Patent Application Publication No. 2001-299676, sentinel lymph nodes at a depth greater than several millimeters from the surface cannot be confirmed.

In general, the temperature of abnormal cells such as cancer cells are around 1° C. higher than that of normal cells. Using this nature, detecting methods disclosed in Japanese Unexamined Patent Application Publication No. 2001-286436, and U.S. Pat. No. 5,445,157, for example, have been proposed wherein infrared light emitted from the portion to be observed in the body cavity is detected so as to measure the temperature of tissue of an organism, and thus abnormal tissue such as cancer cells can be specified.

However, in general, the temperature of a sentinel lymph node is the same as that of the surrounding tissue. Therefore, with the detecting methods disclosed in Japanese Unexamined Patent Application Publication No. 2001-286436, and U.S. Pat. No. 5,445,157, the temperature of the portion to be observed can be measured, but it is difficult to specify sentinel lymph nodes.

### SUMMARY OF THE INVENTION

Accordingly, it is an object of the present invention to provide a sentinel lymph node detecting apparatus and a sentinel lymph node detecting method, wherein the accurate position of a sentinel lymph node can be detected (identified) with the burden placed on a patient such as laparotomy or the like being reduced.

It is another object of the present invention to provide a sentinel lymph node detecting apparatus and a sentinel lymph node detecting method, wherein even deeper sentinel lymph nodes can be detected (identified).

It is another object of the present invention to provide a sentinel lymph node detecting apparatus and a sentinel lymph node detecting method, wherein sentinel lymph nodes at narrow portions in the body cavity, which cannot be readily detected by frontal views, can be detected (identified).

It is another object of the present invention to provide a sentinel lymph node detecting apparatus and a sentinel lymph node detecting method, wherein deep sentinel lymph nodes can be detected, and sentinel lymph nodes at various depths can be detected (identified).

It is yet another object of the present invention to provide a sentinel lymph node detecting apparatus and a sentinel lymph node detecting method, wherein a sentinel lymph node at a desired depth-wise position can be detected (identified).

According to a first aspect of the present invention, a sentinel lymph node detecting apparatus according to the present invention comprises fluctuating magnetic field generating means for vibrating ferrofluid, which has been accumulated in a sentinel lymph node around an affected portion beforehand, by the fluctuation of the magnetic field, so that the ferrofluid is heated, endoscope imaging means for taking endoscope images around the affected portion, temperature change imaging means for taking images of the change in temperature around the affected portion which has been heated due to the fluctuation of the magnetic field generated by the fluctuating magnetic field generating means, and superimposing means for superimposing a temperature-change image obtained by the temperature change imaging means on an endoscope image obtained by the endoscope imaging means.

According to a second aspect of the present invention, a sentinel lymph node detecting method uses a sentinel lymph node detecting apparatus which comprises fluctuating magnetic field generating means for vibrating ferrofluid, which has been accumulated in a sentinel lymph node around an affected portion beforehand, by the fluctuation of the magnetic field, so that the ferrofluid is heated, endoscope imaging means for taking endoscope images around the affected portion, and temperature change imaging means for taking images of the change in temperature around the affected portion which has been heated due to the fluctuation of the magnetic field generated by the fluctuating magnetic field generating means, wherein a temperature-change image obtained by the temperature change imaging means is superimposed on an endoscope image obtained by the endoscope imaging means, so as to identify the position of the sentinel lymph node.

According to a third aspect of the present invention, a sentinel lymph node detecting apparatus comprises a pulse light source for casting pulse light for generating the change in ultrasonic signals with regard to time, occurring from dye due to the optoacoustic effect from absorption of light with a specific wavelength for the dye, light guide means for guiding the pulse light around an affected portion into which the dye has been injected beforehand, a detector which is disposed at a position close to the output end of the light guide means, and detects the ultrasonic signals, and output means for outputting presence or absence of the dye, or the density of the dye, based upon output signals from the detector.

According to a fourth aspect of the present invention, a sentinel lymph node detecting apparatus comprises a light source for exciting fluorescent dye which has been injected into a sentinel lymph node around an affected portion beforehand, an endoscope having a light guide for guiding illumination light from the light source into the body cavity, imaging means for observing fluorescence from the fluorescent dye, which is disposed on the tip of the endoscope, and illumination angle adjusting means for adjusting the illumination angle of the illumination light into the body cavity, which is disposed between the light guide and the body cavity.

According to a fifth aspect of the present invention, a sentinel lymph node detecting apparatus comprises a light source which alternately casts light for exciting a material which has been injected around an affected portion and emits fluorescence when combined with the affected portion, and white light as illumination light, an endoscope for outputting the illumination light from the light source via a light guide, imaging means for observing fluorescence from the material, which is disposed on the tip of the endoscope, recording means for recording reflected-light images and fluorescence images, synchronously with alternating casting of light from the light source, and image synthesizing means for superimposing the fluorescence image and the reflected-light image, which are recorded in the recording means, and displaying the synthesized image.

According to a sixth aspect of the present invention, a sentinel lymph node detecting method comprises a first step wherein a dye which absorbs light with a specific wavelength is injected around affected tissue beforehand, a second step wherein pulse light which generates the change over time in ultrasonic signals occurring in the dye due to the optoacoustic effect from light with the above wavelength, is cast on organic tissue to be observed around the affected tissue, via light guiding means, and a third step wherein presence or absence of the dye, or the density of the dye, is output based upon output signals from a detector which is disposed at a position close to the output end of the light guide means, and detects the ultrasonic signals.

Other features and advantages of the present invention will become apparent from the following description.

### BRIEF DESCRIPTION OF THE DRAWINGS

FIG. 1 is an overall configuration diagram which illustrates a sentinel lymph node detecting apparatus according to a first embodiment of the present invention;

FIG. 2 is an explanatory diagram which illustrates a configuration of the fluctuating magnetic field generating device shown in FIG. 1;

FIG. 3 is a schematic diagram which illustrates a scene of the tip of an inserting portion of an endoscope wherein ferrofluid is being locally injected;

FIG. 4A is an explanatory diagram which illustrates a computer color-enhanced image obtained by the sentinel lymph node detecting apparatus shown in FIG. 1;

FIG. 4B is an explanatory diagram which illustrates an endoscope image obtained by the sentinel lymph node detecting apparatus shown in FIG. 1;

FIG. 4C is an explanatory diagram which illustrates an endoscope infrared image synthesized by superimposing the image shown in FIG. 4A on the image shown in FIG. 4B;

FIG. 5 is a schematic diagram which illustrates a scene of the tip of the inserting portion of the endoscope wherein a sentinel lymph node situated behind the wall of the body cavity such as the stomach is being detected (identified);

FIG. 6 is a schematic diagram which illustrates a scene of the tip of the inserting portion of the endoscope wherein a tracer left in the affected tissue and affected portion is being removed;

FIG. 7 is a schematic diagram which illustrates a scene of the tip of the inserting portion of the endoscope wherein a tracer is being locally injected into an affected portion;

FIG. 8 is an overall configuration diagram which illustrates an sentinel lymph node detecting apparatus according to a second embodiment of the present invention;

FIG. 9 is an explanatory diagram which illustrates a first modification of the probe;

FIG. 10 is an explanatory diagram which illustrates a second modification of the probe;

FIG. 11 is an explanatory diagram which illustrates a third modification of the probe;

FIG. 12 is an explanatory diagram which illustrates a fourth modification of the probe;

FIG. 13 is a principal component cross-sectional view which illustrates a modification of an aspiration biopsy needle shown in FIG. 12;

FIG. 14 is an explanatory diagram which illustrates a probe tip portion on which an opening cap is mounted;

FIG. 15 is a configuration diagram which illustrates a probe of a sentinel lymph node detecting apparatus according to a third embodiment of the present invention;

FIG. 16 is a circuit block diagram which illustrates a microwave detecting circuit for the probe shown in FIG. 15;

FIG. 17 is an overall configuration diagram which illustrates a sentinel lymph node detecting apparatus according to a fourth embodiment of the present invention;

FIG. 18 is a configuration diagram which illustrates a sentinel lymph node detecting apparatus according to a fifth embodiment of the present invention;

FIG. 19 is a chart which illustrates an example of how intensity signals of ultrasonic waves change corresponding to elapsing of time with the sentinel lymph node detecting apparatus according to the fifth embodiment of the present invention;

FIG. 20 is a diagram for describing a configuration example of a sentinel lymph node detecting apparatus according to a modification of the fifth embodiment of the present invention;

FIG. 21 is a configuration diagram which illustrates a sentinel lymph node detecting apparatus according to a sixth embodiment of the present invention;

FIG. 22 is an explanatory diagram which illustrates a configuration of a filter wheel according to the sixth embodiment of the present invention;

FIG. 23 is a light-transmission characteristic diagram for each filter according to the sixth embodiment of the present invention;

FIG. 24 is a configuration diagram which illustrates a sentinel lymph node detecting apparatus according to a seventh embodiment of the present invention;

FIG. 25 is a configuration diagram which illustrates a sentinel lymph node detecting apparatus according to an eighth embodiment of the present invention; and

FIG. 26 is a light-transmission characteristic diagram for each filter according to the eighth embodiment of the present invention.

### DESCRIPTION OF THE PREFERRED EMBODIMENTS

Referring to the drawings, embodiments of the present invention will be described below.

(First Embodiment)

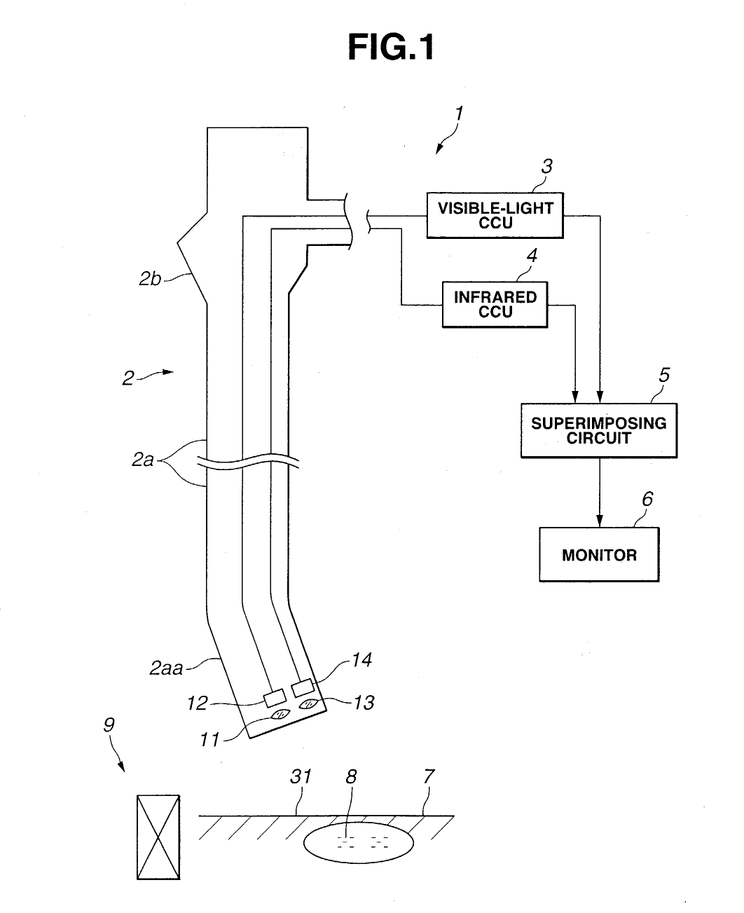

FIGS. 1 through 7 are diagrams for describing a sentinel lymph node detecting apparatus according to a first embodiment of the present invention.

FIG. 1 is an overall configuration diagram which illustrates a sentinel lymph node detecting apparatus having a configuration of the first embodiment according to the present invention.

As shown in FIG. 1, a sentinel lymph node detecting apparatus 1 having the configuration of the first embodiment according to the present invention generally comprises a flexible endoscope 2 (which will be simply referred to as “endoscope” hereafter) including an inserting portion 2a with a small diameter which can be inserted into the body cavity, a visible-light CCU (camera control unit) 3 and infrared-light CCU (camera control unit) 4, which can be detachably connected to the endoscope 2, a superimposing circuit 5 for superimposing an image output from the infrared-light CCU 4 on an image output from the visible-light CCU 3, and a monitor 6 for displaying the superimposed image from the superimposing circuit 5. Furthermore, a fluctuating magnetic field generating device 9 is provided to the sentinel lymph node detecting apparatus 1 for generating a fluctuating magnetic field for vibrating ferrofluid 8 which has been accumulated in a sentinel lymph node 7 beforehand as a tracer so as to generate heat. The configuration of the fluctuating magnetic field generating device 9 will be described later.

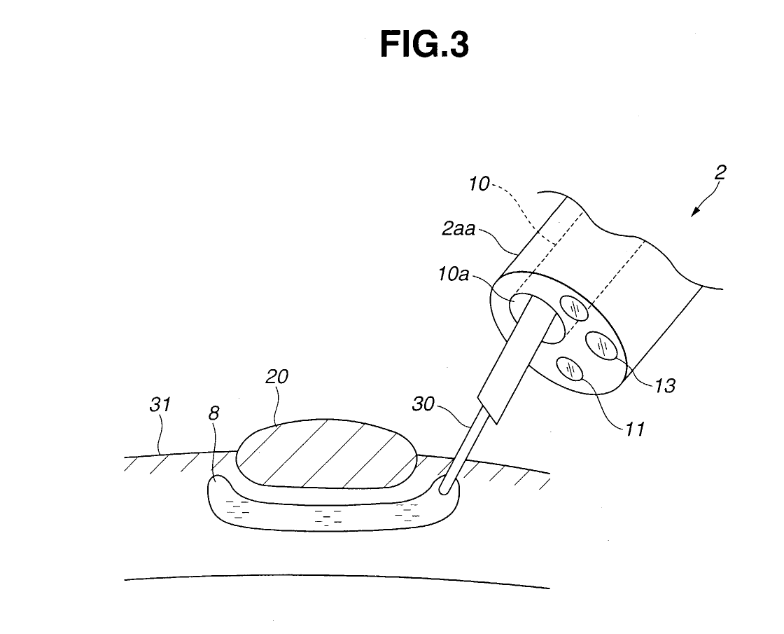

A surgical instrument insertion opening, which is not shown in the drawings, is provided around the end of an operation unit 2b of the endoscope 2 for inserting a surgical instrument such as an injection needle or the like. The surgical instrument insertion opening leads to a surgical instrument inserting channel 10 which will be described later, in the interior of the endoscope. The tip of the surgical instrument is protruded from a channel opening 10a formed at a tip portion 2aa of the inserting portion 2 through the surgical instrument inserting channel 10 inside by inserting a surgical instrument into the surgical instrument insertion opening, so that biopsy (tissue sampling) can be performed (see FIG. 3).

Note that with the present embodiment, an injection needle is inserted from the surgical instrument insertion opening of the endoscope operation unit 2b, and the tip of the injection needle is protruded from the channel opening 10a of the surgical instrument inserting channel 10 as described later, so that ferrofluid 8 is locally injected around affected portions such as cancer tumors. The ferrofluid 8 which has been locally injected around the affected portion migrates from the injected portion to a lymph vessel, reaches a lymph node which is first reached, i.e., the sentinel lymph node 7, and is accumulated in the sentinel lymph node 7.

Furthermore, a light guide which is not shown in the drawings is inserted and disposed within the inserting portion 2a or the like of the endoscope 2. White light is supplied to the tip of the light guide from a light source device which is not shown in the drawings by the endoscope 2, detachably connected to the light source device. The white light guided through the light guide lights up affected portions and so forth in the body from an illuminating optical system which is provided at the inserting portion tip 2aa, that is not shown in the drawings.

A visible light (normal observation) object optical system 11 is disposed neighboring the illuminating optical system at the inserting portion tip 2aa of the endoscope 2, and also a visible-light CCD 12 is provided at the image formation position of the visible-light object optical system 11 as a visible-light imaging device. The endoscope 2 is detachably connected to the visible-light CCU 3, so that extending signal lines of the visible-light CCD 12 are connected to the visible-light CCU 3. The visible-light CCD 12 is driven by power for the CCD and CCD driving pulses being transmitted from the visible-light CCU 3 via the signal lines, generates image signals by taking images (photo-electric conversion) of an image-formed object with visible light, and outputs these to the visible-light CCU 3.

The visible-light CCU 3 performs signal processing for image signals from the visible-light CCD 12 so as to generate standard video signals. The visible-light CCU 3 outputs the video signals to the monitor 6 via the superimposing circuit 5, and the endoscope image taken with visible light is displayed on a display screen of the monitor 6.

Furthermore, with the endoscope 2, an infrared (temperature distribution detecting) object optical system 13 which passes infrared light is disposed neighboring the visible-light object optical system 11 at the inserting portion tip 2aa, and also an infrared sensor (micro-bolometer array device) 14 is provided at the image formation position of the infrared object optical system 13 as an infrared imaging device. The infrared object optical system 13 is made up of lenses formed of zinc selenium or the like, which transmits infrared light.

On the other hand, the infrared sensor 14 (micro-bolometer array device) is an arrangement wherein miniaturized bolometers employing thermistors are two-dimensionally arrayed, and the arrayed bolometers are vacuum-sealed, for example. Accordingly, the infrared sensor 14 is a sensor which can obtain two-dimensional information with regard to infrared light, i.e., image information with regard to infrared light without cooling.

The bolometer for being employed in the infrared sensor 14 measures the temperature of a radiant energy source using the nature of the change in resistance due to temperature increase. With the present embodiment, the infrared sensor 14 employs a thermistor with high sensitivity for temperature change as a bolometer. Thus, the infrared sensor forms a cooling-free micro-bolometer array device which can obtain the temperature distribution image information with regard to the object by miniaturizing each bolometer for being employed (that is to say, employing a micro-bolometer), and two-dimensionally disposing multiple micro-bolometers.

The cooling-free infrared sensor 14 can obtain the high resolution greater than 70,000 pixels, for example, even in the event of employing a configuration with a small size. That is to say, the present infrared sensor 14 can obtain a computer color-enhanced infrared image as a temperature distribution image with a resolution far higher than an arrangement employing infrared transmission fibers.

Also, the present infrared sensor 14 has the advantage of obtaining a computer color-enhanced infrared image as a two-dimensional temperature distribution image with neither contact nor cooling. Moreover, using the cooling-free infrared sensor 14 enables measurement with high precision around 0.1° C., which is equivalent to the temperature noise. Note that the infrared sensor 14 can detect light in the range of 7 μm through 14 μm. Thus, the infrared object optical system 13 employs a material which transmits light at least in a part of wavelength range of 7 μm through 14 μm. The configuration of the present embodiment employs zinc selenium.

With the infrared object optical system 13, which is not shown in the drawings, infrared light lens holding members such as an interval ring for defining a lens interval, a lens frame for holding lens, and so forth, have been subjected to matte processing. Thus, the infrared object optical system 13 is configured so as to reduce noise due to reflection and radiation of infrared light.

As described above, the infrared sensor 14 has a configuration wherein a great number of micro-bolometer elements are two-dimensionally disposed. Also, the infrared sensor 14 has a switching circuit such as a multiplexer at the back side of the infrared detecting face thereof. Accordingly, the infrared sensor 14 accesses each micro-bolometer element via the switching circuit. Thus, the infrared sensor 14 can output signals detected by each micro-bolometer element with a small number of output terminals. Note that the infrared sensor 14 is not restricted to an arrangement employing a thermistor, but an arrangement employing barretters with a small size (which is formed using a extra-fine platinum wire employed in temperature measurement) may be made, for example.

The endoscope 2 is detachably connected to the infrared CCU 4, so that extending signal lines from the infrared sensor 14 are connected to the infrared CCU 4. The infrared CCU 4 transmits power for the sensor and sensor driving pulse signals to the infrared sensor 14 via the signal lines so that the infrared sensor 14 is driven, and detected infrared light is converted into electric signals which are output to the infrared CCU 4 as two-dimensional information with regard to infrared light.

The infrared CCU 4 performs signal processing for electric signals from the infrared sensor 14 so as to generate video signals for a computer color-enhanced infrared image as a temperature distribution image corresponding to the signal intensity, which is output to the superimposing circuit 5.

The superimposing circuit 5 generates video signals for an endoscope infrared image wherein video signals from the infrared CCU 4 are superimposed on video signals from the visible-light CCU 3, and outputs to the monitor 6.

With the present embodiment, the ferrofluid 8 which has been accumulated in the sentinel lymph node 7 beforehand is heated by vibration due to a fluctuating magnetic field generated by the fluctuating magnetic field generating device 9 so that the change in temperature occurs near affected portions such as cancer tumor portions. The change in temperature is detected by the infrared sensor 14 so as to obtain a computer color-enhanced infrared image. The superimposing circuit 5 superimposes the computer color-enhanced infrared image on an ordinary endoscope image which has been taken by the visible-light CCD 12 with visible light, so that the position of the sentinel lymph node 7 is identified.

Now, a configuration of the fluctuating magnetic field generating device 9 will be described.

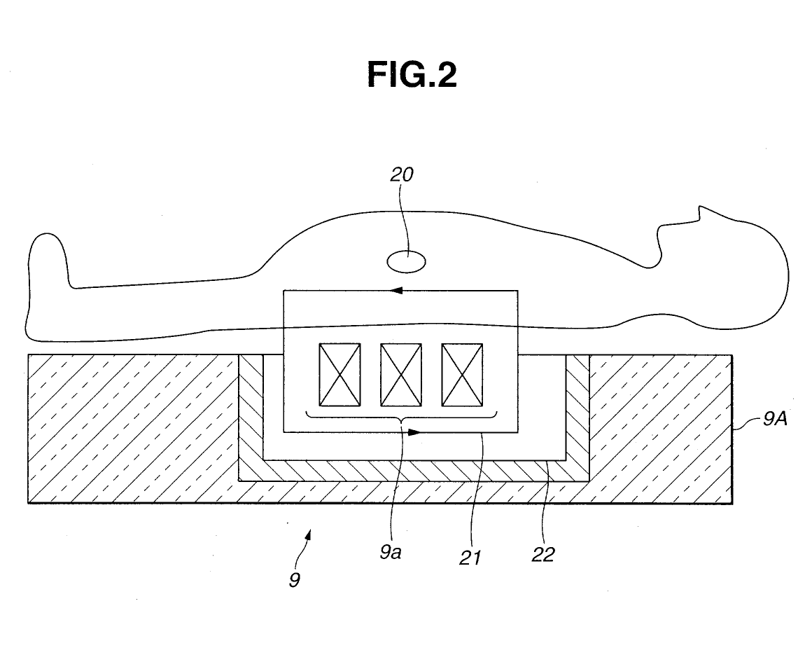

FIG. 2 is an explanatory diagram which illustrates a configuration of the fluctuating magnetic field generating device.

As shown in FIG. 2, with the fluctuating magnetic field generating device 9, multiple magnetic coils 9a are disposed at a main unit 9A formed of an insulator. The magnetic coil 9a generates a fluctuating magnetic field near an affected portion 20 of a patient by changing an alternating magnetic field 21 at a predetermined frequency.

Furthermore, a magnetic shield 22 is provided to the fluctuating magnetic field generating device 9 so as to cover multiple magnetic coils 9a such that the generated fluctuating magnetic field does not act on portions other than the portions around the affected portion 20 of a patient.

The fluctuating magnetic field generating device 9 wherein multiple magnetic coils 9a are provided within the body 9A is connected to a control unit which is not shown in the drawings, and an electric current is controlled so as to form a fluctuating magnetic field by inverting the polarity of the current or changing the amplitude of the current at a predetermined frequency, for example.

The fluctuating magnetic field generating device 9 vibrates and heats the ferrofluid 8 which has been accumulated in the sentinel lymph node 7 beforehand by the generated fluctuating magnetic field. With the sentinel lymph node detecting apparatus 1, the change in temperature near an affected portion, such as a cancer tumor portion, due to the change in temperature of the ferrofluid 8, is detected by the infrared sensor 14 so as to obtain a computed color-enhanced infrared image.

With the sentinel lymph node detecting apparatus 1 having the above-described configuration, the endoscope inserting portion 2a is inserted into the cavity of a patient, and the inserting portion tip 2aa is guided to the affected portion 20 such as the stomach, by operations performed by a surgeon.

Subsequently, the surgeon inserts an injection needle 30 from a surgical instrument insertion opening of the endoscope operation unit 2b, and protrudes the tip of the injection needle 30 from the channel opening 10a of the surgical instrument inserting channel 10 as shown in FIG. 3. FIG. 3 is a schematic diagram which illustrates a scene of the tip of the insertion portion of the endoscope with ferrofluid being locally injected.

Next, the surgeon inserts an injection needle 30 into a lower portion of the affected portion 20 on the wall of the body cavity 31, and locally injects the ferrofluid 8 around the affected portion while observing endoscope images with visible light obtained by taking images using the visible-light CCD 2 displayed on the monitor 6. The ferrofluid 8 locally injected around the affected portion is then transferred to a lymphatic vessel from the injected portion, reaches the sentinel lymph node 7, after 5 to 15 minutes, and is accumulated in the sentinel lymph node 7.

Subsequently, the surgeon drives the fluctuating magnetic field generating device 9 as shown in FIG. 2, and generates a fluctuating magnetic field near the affected portion 20 of the patient. The ferrofluid 8 which has been accumulated in the sentinel lymph node 7 beforehand is vibrated and heated due to the fluctuating magnetic field generated by the fluctuating magnetic field generating device 9.

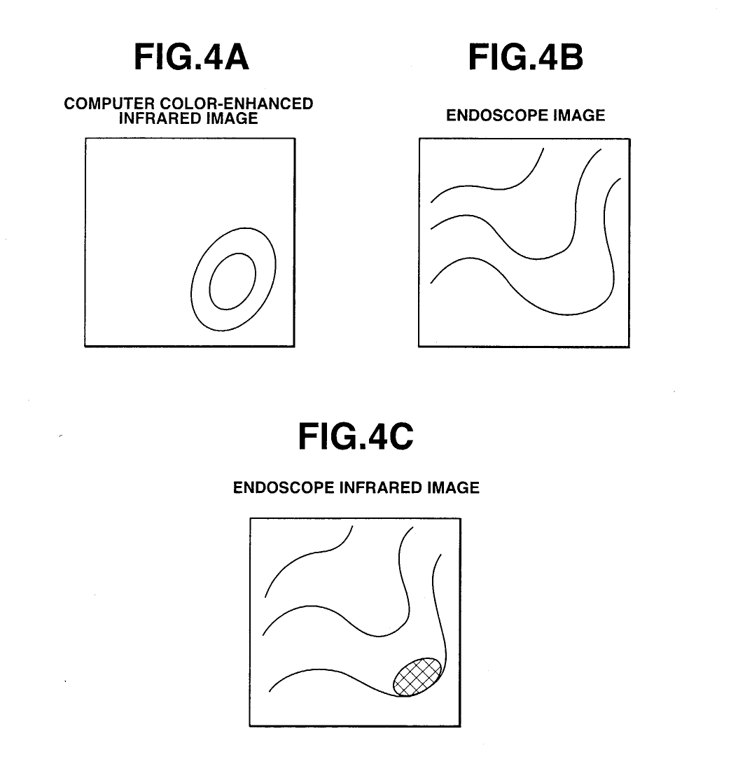

The surgeon obtains an endoscope image of the affected portion 20 as shown in FIG. 4A by taking an image of the affected portion 20 using the visible-light CCD 12, and also obtains a computer color-enhanced infrared image as shown in FIG. 4B by taking an image of the change in temperature near the affected portion. The computer color-enhanced infrared image is superimposed on an endoscope image of the affected portion 20 by the superimposing circuit 5, and an endoscope infrared image is displayed on the display screen of the monitor 6. FIG. 4A is an explanatory diagram which illustrates a computer color-enhanced infrared image obtained by the sentinel lymph node detecting apparatus shown in FIG. 1, FIG. 4B is an explanatory diagram which illustrates an endoscope image obtained by the sentinel lymph node detecting apparatus shown in FIG. 1, and FIG. 4C is an explanatory diagram which illustrates an endoscope infrared image wherein the image shown in FIG. 4A is superimposed on the image shown in FIG. 4B.

Here, in the event that the sentinel lymph node 7 is in the imaging range of the infrared sensor 14, the temperature thereof is higher than that of the surrounding portions due to heating of the ferrofluid 8 accumulated in the sentinel lymph node 7. Accordingly, in the computer color-enhanced image obtained by taking images using the infrared sensor 14, the color tone of the sentinel lymph node 7 is altered.

Accordingly, using the sentinel lymph node detecting apparatus 1, a user can easily recognize the relationship between the position of the affected portion, the position of the internal organ, and the position of the sentinel lymph node 7, from the endoscope infrared image shown in FIG. 4C, and thus can detect (identify) the sentinel lymph node 7. Note that, with the sentinel lymph node detecting apparatus 1, other sentinel lymph nodes 7 can be detected (identified) by obtaining computer color-enhanced infrared image while moving the inserting portion tip 2aa of the endoscope 2 around the affected portion.

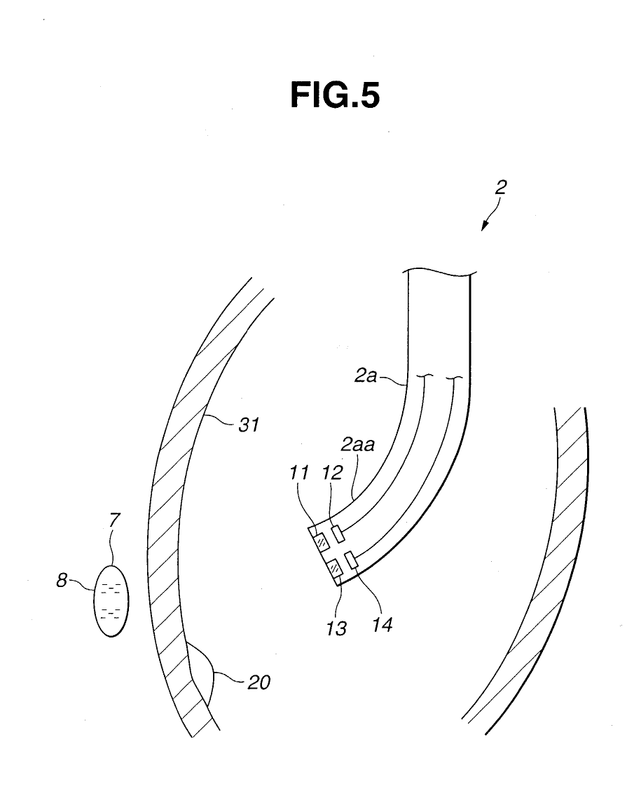

Also, the sentinel lymph node detecting apparatus 1 can detect (identify) the sentinel lymph node 7 by transmission of infrared light even if the sentinel lymph node 7 is behind the wall of the body cavity 31 as shown in FIG. 5.

FIG. 5 is a schematic diagram which illustrates a scene of the tip of the inserting portion of the endoscope with a sentinel lymph node behind the wall of the body cavity such as the stomach, being detected (identified).

In this case, a surgeon can mark the surface of the wall of the body cavity 31 for the detected sentinel lymph node 7 with indocyanine green or the like using the injection needle 30, or can take a tissue sample by inserting the aspiration biopsy needle into the sentinel lymph node 7, while observing endoscope images, not shown in the drawings. Note that the sentinel lymph node detecting apparatus 1 can detect (identify) the sentinel lymph node 7 by transmission of infrared light even with sentinel lymph nodes 7 hidden behind fat, sentinel lymph nodes 7 exhibiting deposit of carbon, and sentinel lymph node behind the body cavity 31.

Note that the ferrofluid 8 as a tracer may be mixed with a dye such as indocyanine green, patent blue, or the like, when using. In this case, the sentinel lymph node detecting apparatus 1 can detect (identify) the sentinel lymph node 7 on the surface of the wall of the body cavity 31 from endoscope images alone.

As a result, the sentinel lymph node detecting apparatus 1 of the present embodiment can identify the accurate position of the sentinel lymph node 7, and has the advantage that the burden placed on a patient such as laparotomy is reduced.

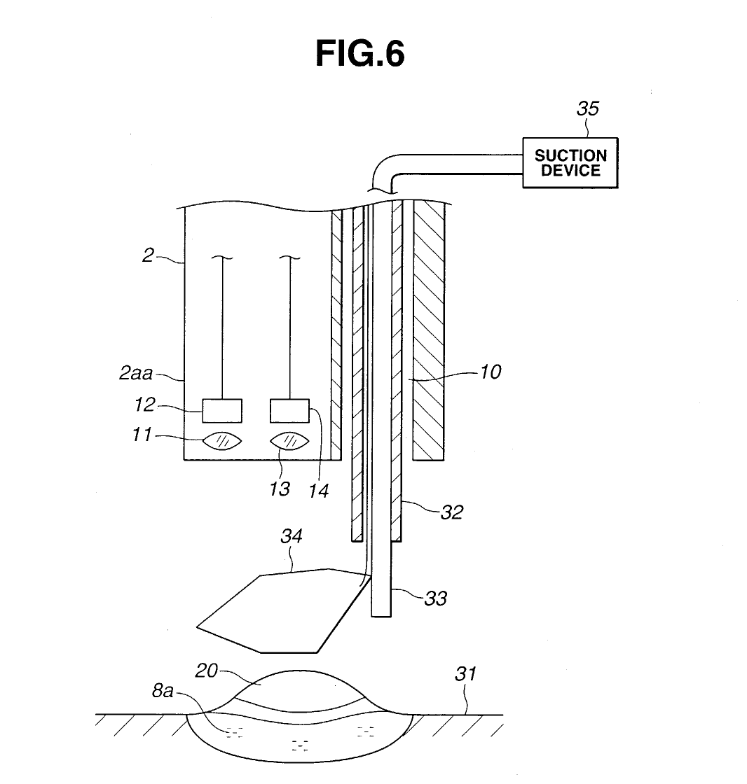

Note that, with the sentinel lymph node detecting apparatus 1, a tracer such as the ferrofluid 8 or the like which has been left around the affected portion 20 causes interference of detection of the sentinel lymph node 7 following identifying of the sentinel lymph node 7. Accordingly, the sentinel lymph node detecting apparatus 1 may have a configuration wherein, following excision of the affected portion 20, the excised tissue and the tracer left around the affected portion are removed as shown in FIG. 6.

FIG. 6 is a schematic diagram which illustrates a scene of the tip of the inserting portion of the endoscope with the tissue of the affected portion and the tracer left around the affected portion, being removed.

As shown in FIG. 6, with the sentinel lymph node detecting apparatus 1, a surgical instrument 32 is inserted into the surgical inserting channel 10 of the endoscope 2, and also a suction catheter 33 and snare 34 are inserted into the inner tube of the surgical instrument 32. The base of the suction catheter 33 is connected to a suction device 35.

Thus, the sentinel lymph node detecting apparatus 1 having the configuration as described above can excise the affected portion 20 with the snare 34 inserted into the surgical instrument 32 of the endoscope inserting portion 2a, and can remove the excised tissue and the tracer 8a left around the affected portion 20 by suctioning using the suction catheter 33. In this state, the sentinel lymph node detecting apparatus 1 can detect (identify) the position of the sentinel lymph node 7.

Thus, the sentinel lymph node detecting apparatus 1 can excise the affected portion 20, and can remove the excised tissue and the tracer 8a left around the affected portion 20, thereby facilitating detection (identification) of the position of the sentinel lymph node 7.

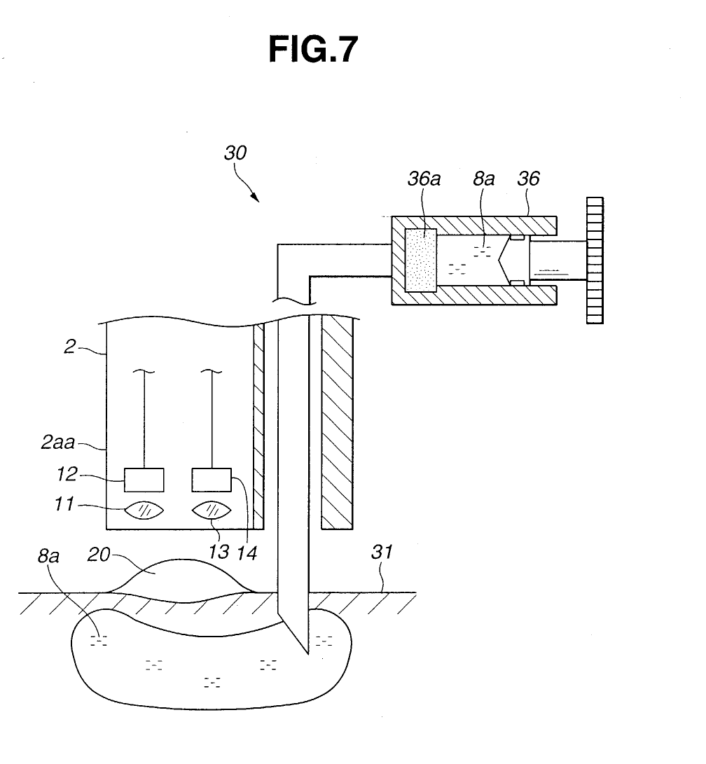

Note that injection needle 30 for locally injecting the tracer 8a is arranged to be connected to an injector as shown in FIG. 7.

FIG. 7 is a schematic diagram which illustrates a scene of the tip of the inserting portion of the endoscope with a tracer being locally injected.

As shown in FIG. 7, the injection needle 30 is arranged so that the base thereof is connected to an injector 36. The injector 36 includes a filter 36a for filtering the tracer 8a into that with a uniform particle size.

Using the injection needle 30 arranged as described above, the sentinel lymph node detecting apparatus 1 identify the position of the sentinel lymph node 7. At this time, a surgeon inserts the endoscope inserting portion 2a into the body cavity, inserts the injection needle 30 around the affected portion 20 on the wall of the body cavity 31, and locally injects the tracer 8a around the affected portion 20 in the state that the tracer 8a has been subjected to filtration by the filter 36a of the injector 36. Thus, the tracer 8a which has been locally injected is filtered into that with a uniform particle size, so a situation wherein the lymph node becomes clogged with the tracer can be avoided, and thus the tracer flows into the sentinel lymph node 7 in a sure manner and is accumulated therein.

Thus, with the sentinel lymph node detecting apparatus 1, the tracer 8a which has been locally injected in the event of identifying the sentinel lymph node 7 flows without the lymph node clogging, thereby enabling the sentinel lymph node 7 to be identified in a sure manner.

(Second Embodiment)

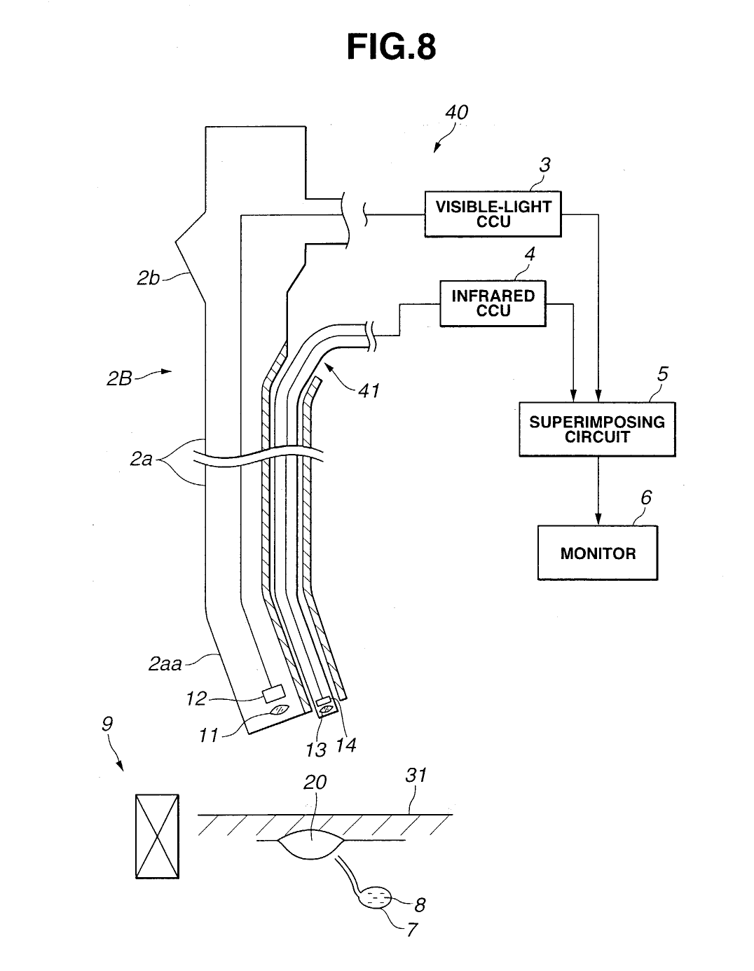

FIGS. 8 through 14 are diagrams for describing a sentinel lymph node detecting apparatus according to a second embodiment of the present invention.

While the above-described first embodiment has a configuration wherein the infrared sensor 14 is disposed on the inserting portion tip 2aa, the second embodiment has a configuration wherein the infrared sensor 14 is disposed on a probe which can be inserted into the surgical instrument inserting channel 10 of the endoscope 2. Other components are generally the same as those of the above-described first embodiment, so the same components will be denoted with the same reference numerals, and description thereof will be omitted.

FIG. 8 is an overall configuration diagram which illustrates a sentinel lymph node detecting apparatus having a configuration according to the second embodiment of the present invention.

As shown in FIG. 8, a sentinel lymph node detecting apparatus 40 according to the second embodiment of the present invention has a configuration wherein the infrared object optical system 13 and the infrared sensor 14, described in the first embodiment, are disposed on a probe 41 which can be inserted into the surgical instrument inserting channel 10 of the endoscope 2B.

The probe 41 is detachably connected to the infrared CCU 4, and the infrared sensor 14 is driven and controlled by the infrared CCU 4. Other components are the same as those described in the above first embodiment, so description will be omitted.

With the sentinel lymph node detecting apparatus 40 as described above, the endoscope inserting portion 2a is inserted into the body cavity of a patient, and the inserting portion tip 2aa is guided to the affected portion 20 such as the stomach, the same as described in the first embodiment.

Subsequently, the surgeon protrudes the tip of the injection needle 30 from the channel opening 10a, the same as described in the first embodiment described above, and the ferrofluid 8 is locally injected as a tracer near the affected portion while observing endoscope images on the monitor 6. The ferrofluid 8 locally injected into the affected portion 20 then reaches the sentinel lymph node 7 following a predetermined time period, and is accumulated therein.

Next, the surgeon drives the fluctuating magnetic field generating device 9, the same as described in the first embodiment described above, so as to generate a fluctuating magnetic field near the affected portion 20 of the patient. The ferrofluid 8 accumulated in the sentinel lymph node 7 is vibrated due to the fluctuating magnetic field generated by the fluctuating magnetic field generating device 9, and is heated.

Subsequently, the surgeon inserts the probe 41 from the surgical instrument insertion opening of the endoscope operation unit 2b, and protrudes the tip of the probe from the channel opening 10a of the surgical instrument inserting channel 10, as shown in FIG. 8. The surgeon then obtains endoscope images of the affected portion 20 by taking images of the affected portion 20 using the visible-light CCD 12 of the endoscope, the same as described in the first embodiment, and also obtains computer color-enhanced infrared images by taking images of the change in temperature near the affected portion using the probe 41.

The computer color-enhanced infrared image is superimposed on the endoscope image of the affected portion 20 by the superimposing circuit 5, the same as described in the first embodiment, so as to be displayed on the display screen on the monitor 6 in a superimposed manner.

As a result, the sentinel lymph node detecting apparatus 40 of this second embodiment can easily take images of the sentinel lymph node 7, even if situated in the body cavity tube with a small diameter, by using the infrared sensor 14 being disposed at the probe 41 with a small diameter, as well as obtaining the same advantages as the first embodiment described above.

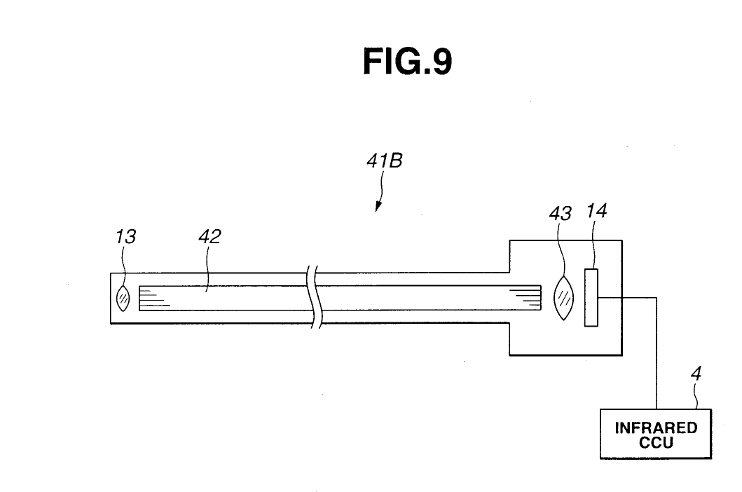

Note that the probe may have a configuration such as shown in FIG. 9.

FIG. 9 is an explanatory diagram which illustrates a first modification of the probe.

As shown in FIG. 9, the probe 41B has a configuration wherein the light input end face of an infrared guide 42 such as a calcogenite fiber, which can guide infrared light, is disposed at the image formation position of the infrared object optical system 13 disposed at the tip of the probe, in a fixed manner. Furthermore, with the probe 41B, a condensing optical system 43 is disposed at the light output end face of the infrared guide 42, and the infrared sensor 14 is disposed at the condensing position of the condensing optical system 43. Thus, with the probe 41B, the diameter of the probe tip can be further reduced.

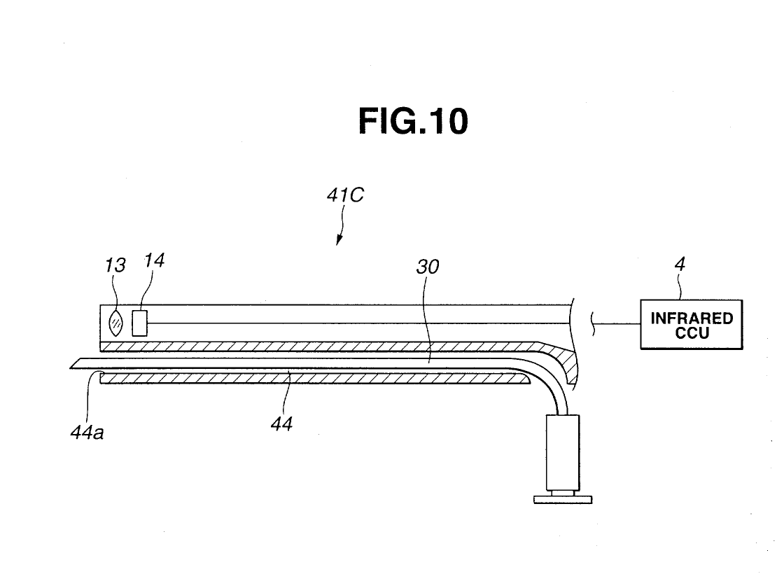

Also, the probe may have a configuration such as shown in FIG. 10.

FIG. 10 is an explanatory diagram which illustrates a second modification of the probe.

As shown in FIG. 10, a surgical instrument inserting channel 44 into which a surgical instrument such as the injection needle 30 can be inserted is provided to the probe 41C, and a channel opening 44a of the channel 44 is formed at the tip of the probe. Thus, the injection needle 30 for marking, or the like, can be inserted into the probe 41C.

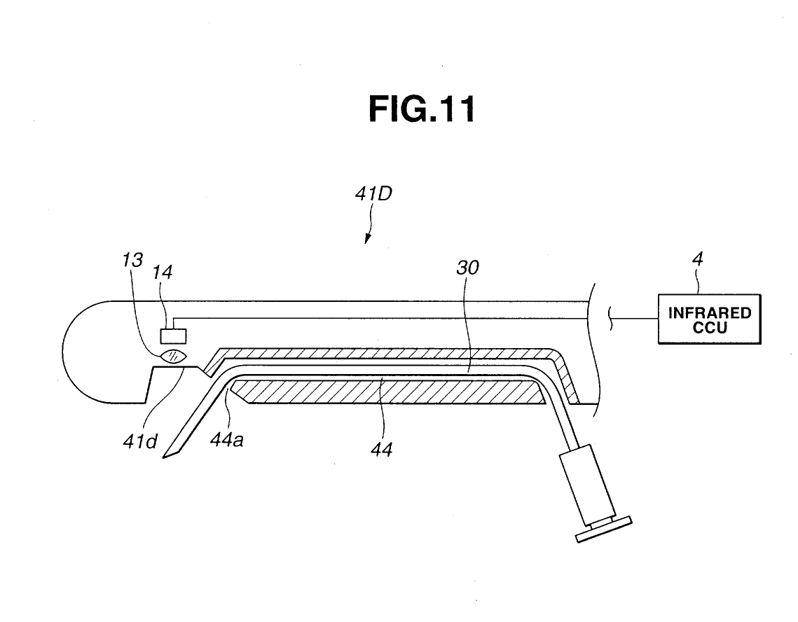

Also, the probe may have a configuration for side-viewing as shown in FIG. 11.

FIG. 11 is an explanatory diagram which illustrates a third modification of the probe.

As shown in FIG. 11, the tip of a probe 41D forms a side-viewing recessed portion 41d, and the infrared object optical system 13 is disposed at the bottom face of the recessed portion 41d in the direction generally orthogonal to the longitudinal direction, and also the infrared sensor 14 is provided at the image formation position of the infrared object optical system 13. Moreover, a surgical instrument inserting channel 44 is provided to the probe 41D, and a channel opening 44a of the channel 44 is formed at the recessed portion 41d.

Thus, using the probe 41D, a user can detect (identify) the position of the sentinel lymph node 7, situated at a narrow portion, which cannot be readily detected by observing the body cavity from the front, and the injection needle 30 for marking, or the like, can be inserted.

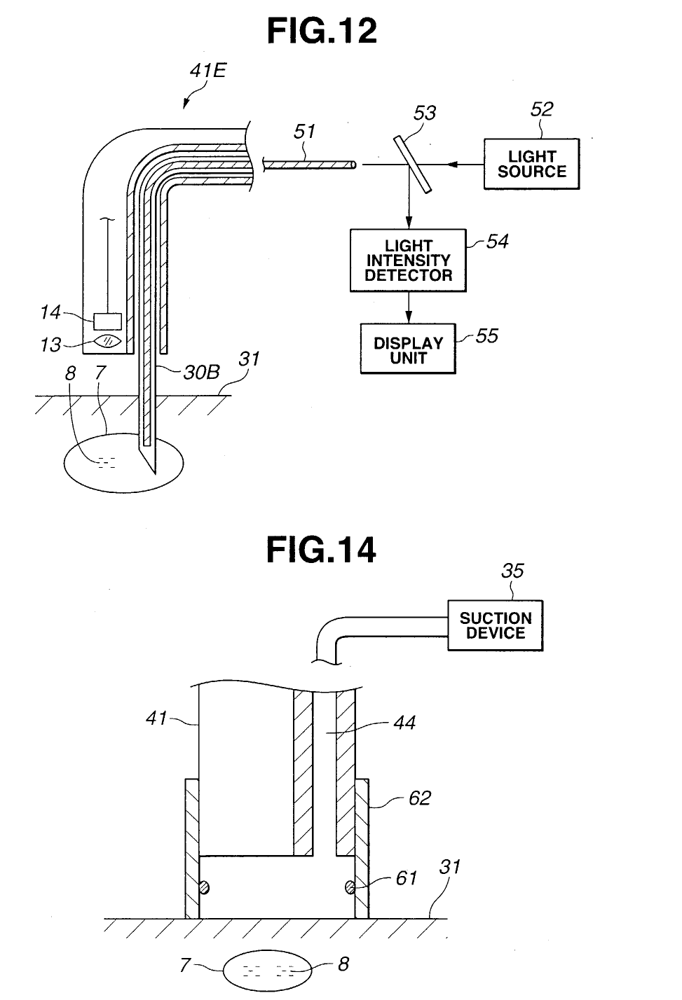

Also, the probe may have a configuration such as shown in FIG. 12.

FIG. 12 is an explanatory diagram which illustrates a third modification of the probe.

As shown in FIG. 12, a probe 41E has a configuration wherein an optical fiber 51 is inserted into an inner tube of the an aspiration biopsy needle 30B which is inserted into the surgical instrument inserting channel 44, and the sentinel lymph node 7 in which the ferrofluid 8 has been accumulated is identified based upon light intensity information obtained from the optical fiber 51.

Furthermore, a light source 52 is provided to the base end of the optical fiber 51 for generating white light or monochromatic light, and also a half mirror 53 is disposed between the optical fiber 51 and the light source 52.

The signal light from the light source 52 is cast into the optical fiber 51 via the half mirror 53, is guided by the optical fiber 51, and is output to the interior of the sentinel lymph node 7 from the tip of the aspiration biopsy needle 30B. The return light such as reflected light, scattered light, and so forth, occurring within the sentinel lymph node 7, returns through the above course in the reverse direction, and reaches the half mirror 53. The return light which has reached the half mirror 53 is reflected, and is input to a light intensity detector 54, so that the quantity of light is detected by the light intensity detector 54.

The light intensity detector 54 outputs the detected light quantity data to a display unit 55, and the display unit 55 displays the light quantity data. Note that the probe 41E has a configuration wherein the position of the sentinel lymph node 7 can be identified, the same as the second embodiment described above.

Using the probe 41E having the configuration as described above, a user can take a tissue sample of the sentinel lymph node 7 within the wall of the body cavity 31 following identifying of the position of the sentinel lymph node 7.

In this case, a surgeon inserts the aspiration biopsy needle 30B into the wall of the body cavity 31, so that the tip of the aspiration biopsy needle 30B reaches the interior of the sentinel lymph node 7. Subsequently, the probe 41E casts signal light from the light source 52 as described above.

At this time, the light quantity of the return light at the tip of the optical fiber 51 is markedly altered according to the presence or absence of the ferrofluid 8. The change in the light quantity of the return light is detected by the light intensity detector 54 via the half mirror 53, and is displayed on the display unit 55. The surgeon can recognize that the aspiration biopsy needle 30B has reached the sentinel lymph node 7 by observing the display state. Note that the display unit 55 may notify the surgeon that the aspiration biopsy needle 30B has reached the sentinel lymph node 7 by sound as well as by displaying on the screen.

Following the aspiration biopsy needle 30B reaching the sentinel lymph node 7, the surgeon extracts the optical fiber 51 from the inner tube of the aspiration biopsy needle 30B, and can take a tissue sample of the sentinel lymph node 7 by suctioning.

Thus, using the probe 41E, a user can identify the depth-wise position of the sentinel lymph node 7 within the wall of the body cavity based upon the reflected light from the optical fiber 51, in particular, in the event of taking a tissue sample of the identified sentinel lymph node 7.

On the other hand, a surgeon must extract the optical fiber 51, which has been inserted into the inner tube of the aspiration biopsy needle 30B used in the probe for identifying the depth of the sentinel lymph node 7, from the inner tube thereof by moving the optical fiber 51 for a long distance when sucking a tissue sample.

Thus, the aspiration biopsy needle 30B used in the probe may have a configuration wherein the moving distance for the optical fiber 51 is reduced so as to improve the operability.

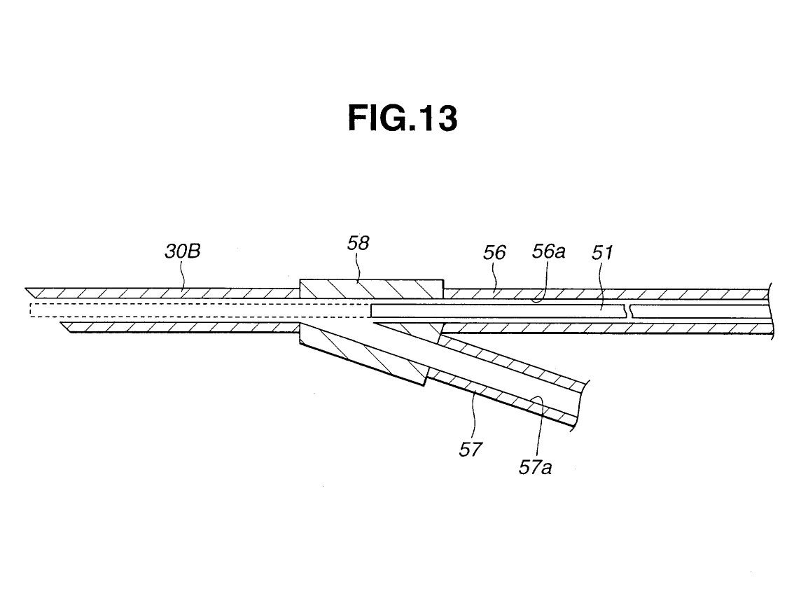

FIG. 13 is a principal component cross-sectional view which illustrates a modification of the aspiration biopsy needle shown in FIG. 12.

As shown in FIG. 13, the aspiration biopsy needle 30B is arranged such that the aspiration biopsy needle 30B is connected to a forked tube 58 of which the probe base side is branched into a fiber inserting tube 56 and a suction tube 57.

The optical fiber 51 can be inserted into the inner tube portion 56a of the fiber inserting tube 56. On the other hand, the suction device 35 can be connected to the inner tube portion 57a of the suction tube 57.

The aspiration biopsy needle 30B having the configuration as described above is inserted up to the position of the sentinel lymph node 7 within the wall of the body cavity 31 in order to take a tissue sample. Subsequently, the surgeon recognizes the position of the sentinel lymph node 7 based upon the reflection of light from the optical fiber 51 with the optical fiber 51 being inserted up to the tip of the aspiration biopsy needle 30B. Following recognition, the surgeon drives the suction device with the optical fiber being retracted up to the forked tube 58, and sucks a tissue sample of the sentinel lymph node 7 through the suction tube 57.

Thus, with the aspiration biopsy needle 30B, it is not necessary that the optical fiber 51 be completely extracted, but rather simply moving the optical fiber 51 up to the position of the forked tube 58 for a short distance enables a tissue sample of the sentinel lymph node 7 to be taken following confirmation of the position of the sentinel lymph node 7.

Also, the probe 41 may have a configuration wherein an opening cap is detachably mounted to the tip of the probe as shown in FIG. 14.

FIG. 14 is an explanatory diagram which illustrates the tip of the probe to which an opening cap is mounted.

As shown in FIG. 14, an opening cap 62 holding a detachable rubber ring 61 within the inner circumference thereof is detachably mounted to the probe 41. Moreover, the suction device 35 is disposed at the base side of the surgical inserting channel 44 of the probe 41. Note that the probe 41 has a configuration wherein the position of the sentinel lymph node 7 can be identified, the same as the second embodiment described above.

With the probe 41 having the configuration as described above, the opening cap 62 is pressed into contact against the wall of the body cavity 31 at which the identified sentinel lymph node 7 is situated, and suctioning is performed by the suction device 35 following identification processing for the position of the sentinel lymph node 7. The probe 41 then performs suctioning of air by the suction device 35, and sucks the sentinel lymph node 7 upward along with the wall of the body cavity 31. The rubber ring 61 is snapped onto the protruded body cavity wall 31 containing the sentinel lymph node 7 by the suctioning action, thereby marking the position of the sentinel lymph node 7. Subsequently, a surgeon can take a tissue sample of the sentinel lymph node 7 by inserting the injection needle 30 into the sentinel lymph node 7 onto which the rubber ring 61 has been snapped.

Thus, using the probe 41, a surgeon can easily mark the sentinel lymph node 7 by sucking the sentinel lymph node 7 and the portions therearound following detecting the sentinel lymph node 7, thereby enabling biopsy to be performed in a sure manner.

(Third Embodiment)

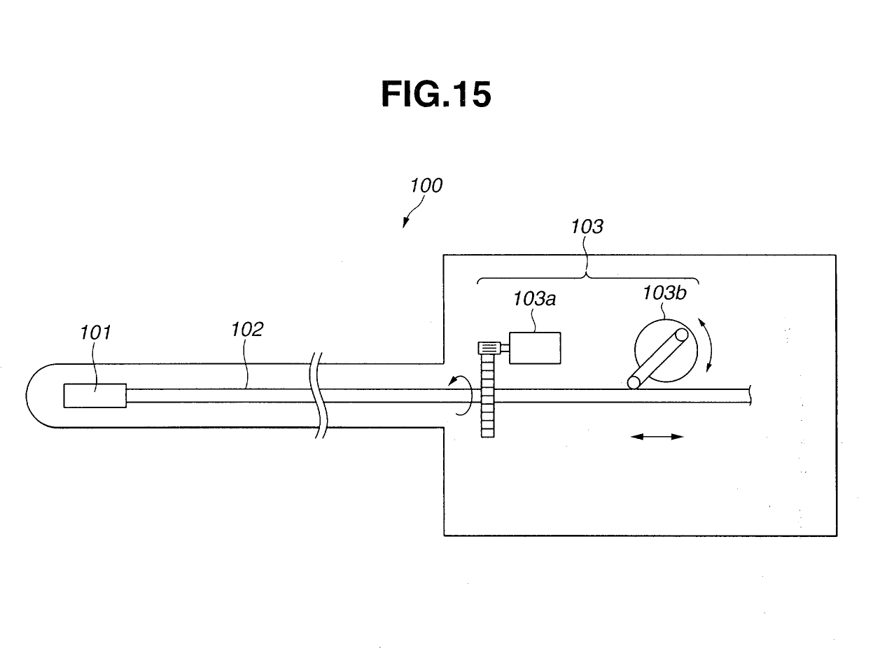

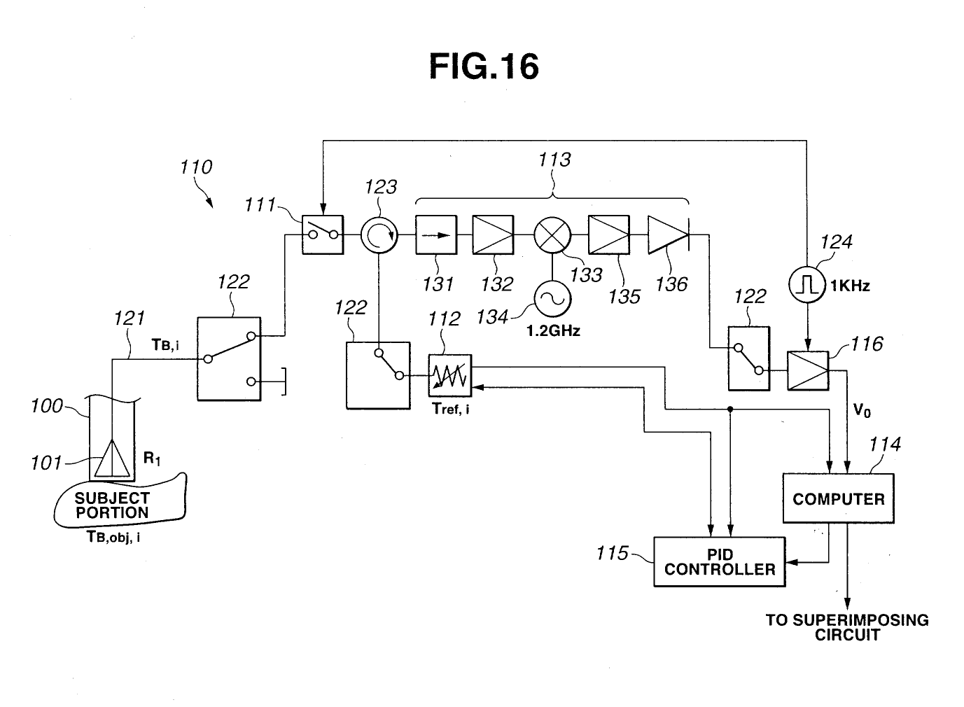

FIGS. 15 and 16 are diagrams for describing a sentinel lymph node detecting apparatus according to a third embodiment of the present invention.

While the first and second embodiments described above employ the infrared sensor 14, this third embodiment employs a microwave antenna. Other components are generally the same as the first embodiment and second embodiment as described above, so description will be omitted, and the same components are denoted with the same reference numerals.

FIG. 15 is a configuration diagram which illustrates a probe of a sentinel lymph node detecting apparatus having a configuration according to the third embodiment of the present invention, and FIG. 16 is a circuit block diagram which illustrates a microwave detecting circuit for the probe shown in FIG. 15.

As shown in FIG. 15, a sentinel lymph node detecting apparatus according to this third embodiment has a configuration wherein a probe 100 including a microwave antenna (which will be simply referred to as “antenna”) made up of a wave guide, instead of the infrared sensor 14, is employed. Note that the probe 100 is used by being inserted into the surgical inserting channel 10 of the endoscope, as described in the second embodiment.

An antenna 101 has a configuration wherein the change in temperature near affected portions such as cancer tumor portions is obtained by detecting microwaves emitted from the ferrofluid 8 accumulated in the sentinel lymph node 7. Note that the ferrofluid 8 accumulated in the sentinel lymph node 7 is heated by being vibrated due to a fluctuating magnetic field generated by the fluctuating magnetic generating device 9, the same as described in the first embodiment.

With the probe 100, the antenna 101 provided at the tip is secured to a shaft 102 rotatably mounted, which can be rotationally driven by a driving unit 103 at the base end.

The driving unit 103 comprises a rotational driving unit 103a for rotating the antenna in an arbitrary manner, and a shifting driving unit 103b for shifting the antenna 101 in the probe longitudinal axis direction. Thus, the antenna 101 can be rotated in an arbitrary manner, and also can be shifted in the probe longitudinal axis direction, thereby enabling helical scanning (radial liner scanning).

Furthermore, the antenna 101 is arranged to be connected to a microwave detecting circuit 110 as shown in FIG. 16.

As shown in FIG. 16, the microwave detecting circuit 110 has a standard configuration comprising the antenna 101, a Dickc switch 111, a reference temperature thermal noise source 112, and a heterodyne receiver 113, and performs a brightness temperature measurement by automatically controlling a PID controller 115 using a computer 114. The computer 114 also generates video signals for a computer color-enhanced image to serve as a temperature distribution image based upon the measured brightness temperature data. Subsequently, the computer 114 outputs generated video signals for the computer color-enhanced image to the superimposing circuit 5 described in the first embodiment.

Now, the configuration of the microwave detecting circuit 110 will be described more specifically.

The microwave detecting circuit 110 is a high sensitive receiver for measuring thermal-noise power emitted from an object. The microwave detecting circuit 110 comprises the heterodyne receiver 113 wherein the Dickc switch 111 is inserted into the input end thereof as a chopper, and a lock-in amplifier 116. The microwave detecting circuit 110 performs signal processing for thermal radiation electric waves (microwaves) received by the antenna 101 following procedures as will be described below. With the microwave detecting circuit 110, thermal radiation electric waves received by the antenna 101 are subjected to waveguide coaxial conversion, and the converted signals are input to the receiver 113 via a low-loss coaxial cable 121, a coaxial switch 122, the Dickc switch 111, and a circulator 123.

The Dickc switch 111 performs switching at 1 kHz so as to observe thermal radiation electric waves from the antenna 101 and the thermal radiation from the reference temperature thermal noise source (which will be referred to as “noise source” hereafter) 112 in an alternating manner, and input to the receiver 113.

The receiver 113 is designed so that the observing frequency is 1.2 GHz, and the band width thereof is 0.4 GHz. The frequency-converted thermal radiation electric waves pass through a square detector 124, the signal components thereof synchronous to 1 kHz are detected and integrated by the lock-in amplifier 116, and are output as a voltage value V₀. The voltage value V₀ is proportional to the difference between the thermal radiation electric waves received by the antenna 101 and the thermal radiation electric waves from the noise source, and the temperature T_{ref.j} of the noise source 112 is automatically controlled so that V₀ is 0. The temperature T_{ref.j} is output as a output value of the microwave detecting circuit 10. Reference numeral 131 denotes an isolator, reference numeral 132 denotes an RF amplifier, reference numeral 133 denotes a mixer, reference numeral 134 denotes an RF source, reference numeral 135 denotes an IF amplifier, and reference numeral 136 denotes a detector.

With the probe 100 having the configuration as described above, the sentinel lymph node detecting apparatus of this third embodiment performs detecting (identification) of the sentinel lymph node 7, the same as the second embodiment described above. In this case, microwaves can transmit up to a body depth greater than that of infrared rays which can only transmit up to a depth near the surface of the organic tissue.

Accordingly, the probe 100 can detect microwaves occurring due to the thermal diffusion of the ferrofluid 8 accumulated in the sentinel lymph node 7 situated within the deep portion of the body, thereby enabling the temperature of the deep portion of the body to be measured.

As a result, the sentinel lymph node detecting apparatus according to this third embodiment can detect (identify) the position of the sentinel lymph node 7 up to the depth greater than that in a case of the first and second embodiment.

(Fourth Embodiment)

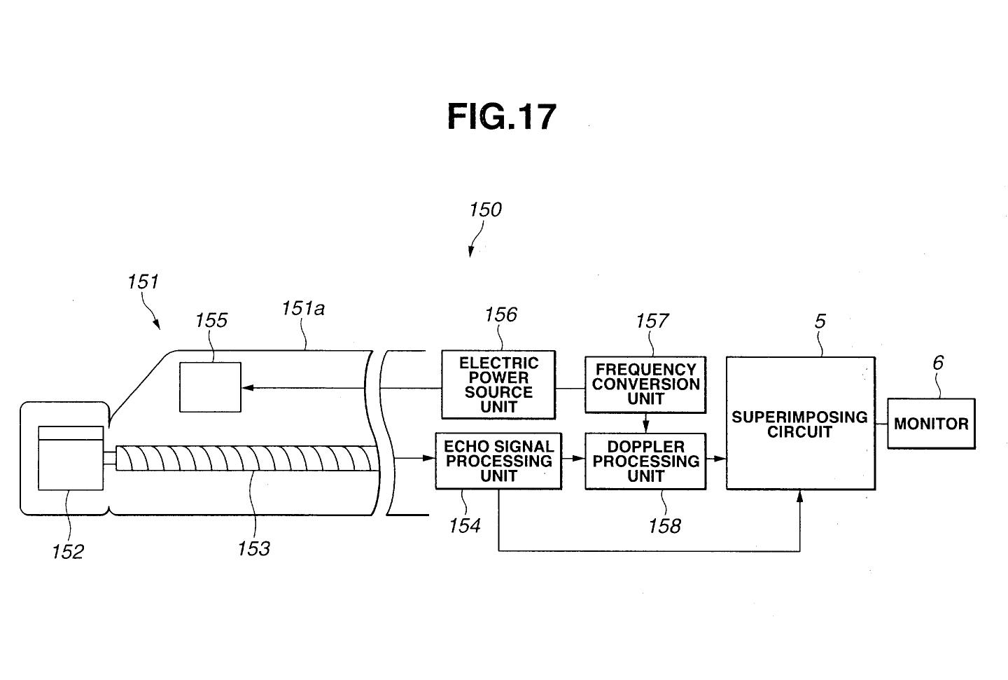

FIG. 17 is an overall configuration diagram which illustrates a sentinel lymph node detecting apparatus according to a fourth embodiment of the present invention.

This fourth embodiment has a configuration wherein identification of the position of the sentinel lymph node is performed using ultrasonic waves.

That is to say, as shown in FIG. 17, a sentinel lymph node detecting apparatus 150 according to the fourth embodiment of the present invention comprises an ultrasonic endoscope 151. With the ultrasonic endoscope 151, an ultrasonic transducer 152 is disposed at an inserting portion tip 151a for transmitting and receiving ultrasonic waves. The ultrasonic transducer 152 is secured to a shaft 153 rotatably mounted, and is rotationally driven by a driving unit which is not shown in the drawings.

With the ultrasonic transducer 152, extending signal lines are inserted into the shaft 153 so as to be connected to an echo signal processing unit 154 provided to the base end. The echo signal processing unit 154 performs signal processing for echo signals received by the ultrasonic transducer 152, and generates video signals for an ultrasonic image which is a two-dimensional tomographic image. The echo signal processing unit 154 outputs video signals for an ultrasonic image generated via a Doppler processing unit which will be described later, to the superimposing circuit 5.

Furthermore, with the ultrasonic endoscope 151, a fluctuating magnetic field generating unit 155 such as an electromagnet, the magnetic field coil 9a, or the like, for generating a fluctuating magnetic field, is provided to the inserting portion tip 151a. The fluctuating magnetic field generating unit 155 vibrates the ferrofluid 8 accumulated in the sentinel lymph node 7, generally the same as the first embodiment described above.

A power source unit 156 supplies a flowing current to the fluctuating magnetic field generating unit 155. The power source unit 156 is connected to the frequency conversion unit 157. The frequency conversion unit 157 controls the frequency of the flowing current so that the fluctuating magnetic generating unit 155 generates a fluctuating magnetic field.

Also, the frequency conversion unit 157 is connected to the Doppler processing unit 158, and the processing frequency of the echo signals received by the ultrasonic transducer 152 are controlled by controlling the Doppler processing unit 158, so as to be synchronized with the frequency of a current supplied from the power source unit 156.

The Doppler processing unit 158 acquires Doppler signals from the ferrofluid 8 vibrating at a predetermined frequency from echo signals received by the ultrasonic transducer 152. Moreover, the Doppler processing unit 158 generates video signals for a Doppler image which is a two-dimensional tomographic image wherein the position of the ferrofluid 8 can be detected based upon the acquired Doppler signals, and outputs to the superimposing circuit 5.

Subsequently, the superimposing circuit 5 superimposes the video signals for the Doppler image from the Doppler processing unit 158 on the video signals for the ultrasonic image from the echo signal processing unit 154 so as to generate image signals for an ultrasonic Doppler image, and outputs the generated image to the monitor 6.

With the sentinel lymph node detecting apparatus 150 having the configuration as described above, the inserting portion of the ultrasonic endoscope 151 is inserted into the body cavity of a patient, and the inserting portion tip 151a is guided to the affected portion 20 within the stomach or the like, the same as described in the first embodiment.

Next, the surgeon drives the fluctuating magnetic field generating unit 155 so as to generate a fluctuating magnetic field toward the affected portion 20 of the patient. The ferrofluid 8 accumulated in the sentinel lymph node 7 is vibrated due to the fluctuating magnetic field generated by the fluctuating magnetic field generating unit 155.

Subsequently, the surgeon begins ultrasonic diagnosis. The sentinel lymph node detecting apparatus 150 obtains ultrasonic images of the affected portion 20 by rotationally driving the ultrasonic transducer 152. At the same time, the sentinel lymph node detecting apparatus 150 obtains Doppler images of the ferrofluid 8 vibrating at a predetermined frequency.

The Doppler image is superimposed on the ultrasonic image of the affected portion 20 by the superimposing circuit 5 so as to display ultrasonic Doppler images on the display screen of the monitor 6.

Accordingly, using the sentinel lymph node detecting apparatus 150, a surgeon can easily recognize the relationship between the position of the affected portion, the position of the internal organ, and the position of the sentinel lymph node 7, based upon the ultrasonic Doppler image wherein a Doppler image has been superimposed on a ultrasonic image, and can detect (identify) the sentinel lymph node 7. Note that other sentinel lymph nodes 7 can be detected (identified) by obtaining ultrasonic Doppler images while moving the inserting portion tip 151a around the affected portion 20.

As a result, the sentinel lymph node detecting apparatus 150 according to this fourth embodiment has the same advantages as the first embodiment described above.

(Fifth Embodiment)

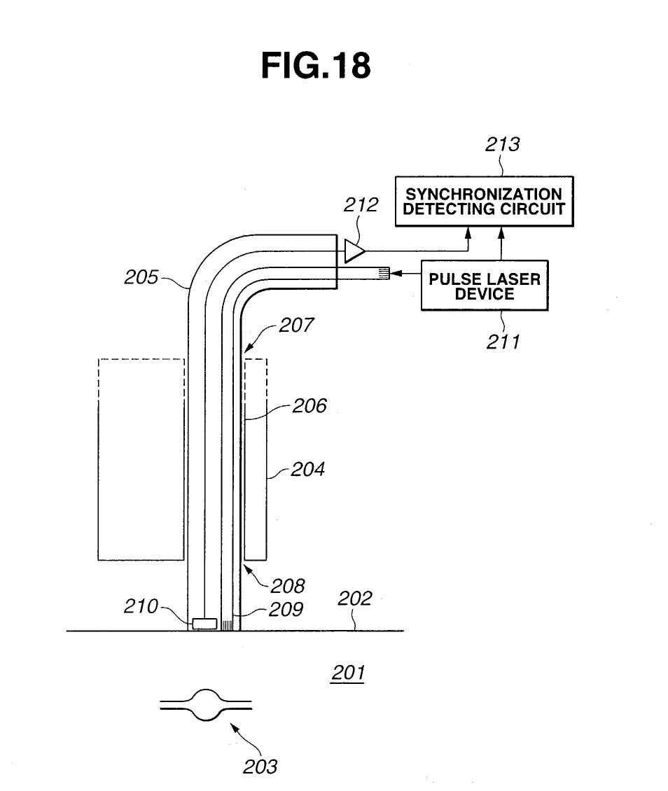

FIGS. 18 through 20 are diagrams for describing a sentinel lymph node detecting apparatus according to a fifth embodiment of the present invention. The sentinel lymph node detecting apparatus according to the present embodiment takes advantage of the nature of the optoacoustic effect.

FIG. 18 is a configuration diagram which illustrates a sentinel lymph node detecting apparatus according to a fifth embodiment. In FIG. 18, reference numeral 201 denotes organic tissue, reference numeral 202 denotes organic tissue surface, reference numeral 203 denotes a sentinel lymph node, and reference numeral 204 denotes an endoscope which includes an imaging device (not shown) such as a charge-coupled device (which will be abbreviated to “CCD” hereafter) or the like, and outputs image signals for displaying images taken by the imaging device on the monitor. Furthermore, reference numeral 205 denotes a probe for being inserted into a channel 206 of the endoscope 204 for surgical instruments, and the probe 205 has an optical fiber 209, which is a light guiding means, inside. The probe 205 is inserted from a forceps opening 207 which is an inserting opening provided to the operating unit of an ordinary endoscope 204, and can be protruded from an opening 208 provided to the tip of the endoscope.

The end of the optical fiber 209 and a piezoelectric device 210 are provided to the tip of the probe 205. The piezoelectric device 210 which is a detector is disposed closely to the output end of the optical fiber 209. The optical fiber 209 guides a pulse laser beam in such a manner wherein a pulse laser beam from the pulse laser device 211 is input from the one end of the base end of the probe 205, and is output from the tip of the probe 205. The pulse laser device 211 is a Q-switch YAG excitation Titanium-sapphire laser device which outputs a pulse laser beam with a pulse width of several ns (nanoseconds), for example. The piezoelectric device 210 which is a transducer receives ultrasonic signals from the organic tissue 201, and outputs the intensity signals of the received ultrasonic signals, as described later. The intensity signals are input to a synchronizing detecting circuit 213 via an amplifier 212. The synchronizing detecting circuit 213 serving as an output means detects timing signals from the pulse laser device 211 and the change in the intensity signals from the amplifier 212 corresponding to time, and outputs signals indicating the presence or absence of dye based upon the change in the intensity signals corresponding to time.

Next, operations of the sentinel lymph node detecting apparatus described above will be described.

First of all, a surgeon locally injects dye, ICG, for example, which absorbs light in a specified wavelength range, around affected portions of a patient, beforehand. Following a predetermined time period for the injected dye to migrate from the injected portions to lymphatic vessels, the surgeon operates the endoscope 204 so that the tip of the probe 205 contacts the surface 202 of the organic tissue 201 while observing affected portions within the body cavity of the patient. The surgeon then operates a switch (not shown) of the pulse laser device 211 so as to detect sentinel lymph nodes.

This ICG has the nature of absorbing near-infrared light in the wavelength range of 800 nm through 900 nm (nanometers). Conversely, the organic tissue 201 itself does not have the nature absorbing near-infrared light in the wavelength range of 800 nm through 900 nm (nanometers).

The pulse laser device 211 casts a pulse laser beam with a wavelength, which the ICG absorbs as described above.

The surgeon turns on the pulse laser device 211. The pulse laser device 211 then outputs a pulse laser beam. The pulse laser beam output from the pulse laser device 211 is cast on the light input end face of the optical fiber 209. The pulse laser beam is then output from the output end of the optical fiber 209 via the interior of the optical fiber 209.

The output pulse laser beam diffuse from the surface 202 near the affected portion into the interior of the organic tissue 201. Upon the ICG which has been injected beforehand receiving a pulse laser beam, the ICG absorbs the light, and generates ultrasonic signals due to the thermoelastic effect. (which is referred to as “optoacoustic effect”).

The generated ultrasonic signals are detected by the piezoelectric device 210. The intensity signals of the detected ultrasonic signals are amplified by the amplifier 212, and are input to the synchronizing detecting circuit 213. The synchronizing detecting circuit 213 detects the presence or absence of ultrasonic signals having a predetermined amplitude or a predetermined width of change, from the piezoelectric device 210 following output of the pulse laser beam from the pulse laser device 211.

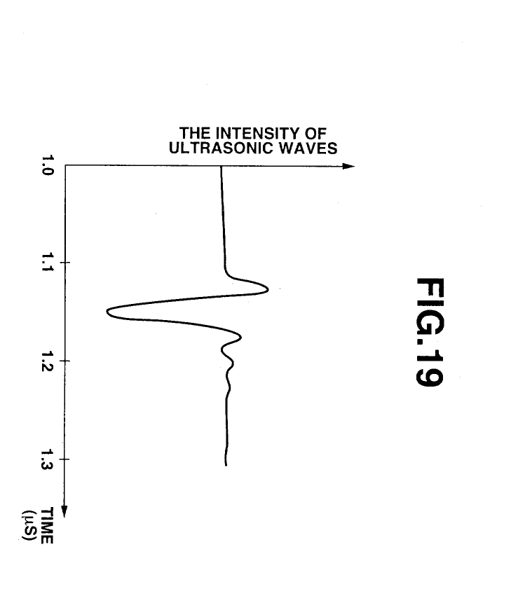

FIG. 19 is a chart which indicates an example of the change in the intensity signals of the ultrasonic signals received by the piezoelectric device 210 over time. The vertical axis indicates the intensity of the ultrasonic signals, and the horizontal axis indicates time elapsing from the pulse laser device 211 outputting a pulse laser beam.

In the example shown in FIG. 19, upon the pulse laser device 211 outputting at 0.0 second, the piezoelectric device 210 receives ultrasonic signals after approximately 1.1 μs (microseconds).

Following output of the pulse laser beam, the intensity of the ultrasonic signals markedly changes generally between 1.1 μs and 1.2 μs (microseconds). Accordingly, judgment can be made that a sentinel lymph node is situated in front of the tip of the probe 205 in the event that the signal intensity indicates a change greater than a predetermined width of change.

While, in this case, judgment is made that there is a sentinel lymph node in the event of detecting the change greater than a predetermined width of change, an arrangement may be made wherein the density of ICG is detected based upon the intensity of the ultrasonic signals. Also, in the event that the change in the ultrasonic signals is equal to or less than the predetermined value, judgment is made that there is no sentinel lymph node.

Accordingly, with the above-described apparatus, light with a predetermined wavelength is cast, and in the event that there is ICG in front of the tip of the probe 205, i.e., there is a sentinel lymph node, the signal intensity of the ultrasonic signals increases due to the optoacoustic effect, thereby enabling the sentinel lymph node to be detected.

Note that the input laser beam should have a pulse width wherein the intensity signals of ultrasonic signals occurring due to the optoacoustic effect changes on the time-axis, and the synchronizing detecting circuit which is a detecting device can detect the presence of ICG.

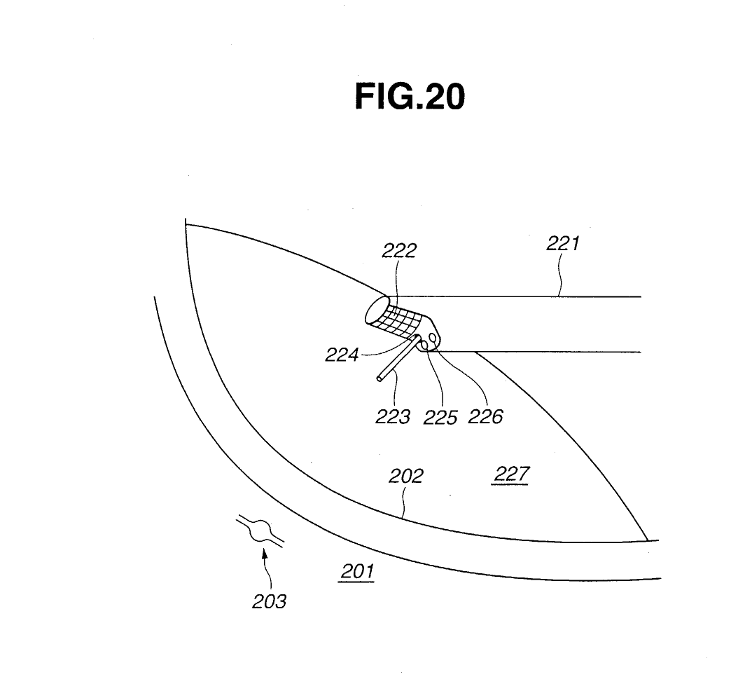

Now, a modification of the fifth embodiment will be described with reference to FIG. 20.

FIG. 20 is a diagram for describing a configuration of a sentinel lymph node detecting apparatus according to a modification of the fifth embodiment.

In FIG. 20, reference numeral 201 denotes organic tissue, reference numeral 202 denotes an organic tissue surface, and reference numeral 203 denotes a sentinel lymph node. Reference numeral 221 denotes an endoscope inserting portion, and reference numeral 222 denotes a piezoelectric element array which is a detector. Reference numeral 223 is an optical fiber. The optical fiber 223 passes through the channel contained in the endoscope inserting portion 221, and the tip thereof is protruded from an opening 224 provided to the tip of the endoscope inserting portion 221. The output end at the tip of the optical fiber 223 is disposed near the piezoelectric element array 222.

Reference numeral 225 denotes an illumination window, and reference numeral 226 denotes an observing window. The reflected light of the light output from the illumination window 225 is input to an imaging device (not shown) via the observing window 226. Thus, the sentinel lymph node detecting apparatus can obtain image signals for the portion to be observed. In the event of using the sentinel lymph node detecting apparatus as an ordinary endoscope, the reflected light output from the illumination window 225 is converted into image signals by the imaging device and images are displayed on a monitor device.

The pulse laser device 211 described in FIG. 18 outputs a pulse laser beam. The pulse laser beam is input from one end of the optical fiber 223, and is output from the output end which is the other end of the optical fiber 223 on the tip side of the endoscope inserting portion 221 toward the portion around the affected portion.

The piezoelectric element array 222 has a configuration wherein multiple piezoelectric elements are disposed in an array shape. The piezoelectric element array 222 two-dimensionally detects ultrasonic signals generated by ICG due to the optoacoustic effect. As a result, the sentinel lymph node detecting apparatus generates two-dimensional images based upon the ultrasonic signals.

On the other hand, water is positioned between the surface 202 of the organic tissue 201 and the piezoelectric element array 222. Ultrasonic signals have the nature of the attenuation thereof being great in air. Accordingly, in the event of detecting sentinel lymph nodes, the surgeon disposes water 227 on the surface of the object 1 so that the water 227 lies between the piezoelectric element array 222 and the organic tissue 201 which is the object to be observed.

Subsequently, the surgeon operates a predetermined switch (not shown) so that a pulse laser beam is cast on the portion around the affected portion via the optical fiber 223. As a result, with the sentinel lymph node detecting apparatus, the ultrasonic signals generated by ICG are received by the piezoelectric element array 222, thereby obtaining the position of the ICG as a two-dimensional image based upon the received ultrasonic signals.

As described above, the present embodiment employs the optoacoustic effect, thereby detecting a sentinel lymph node within the organic tissue at a position up to the depth greater than that in a case of a conventional arrangement.

(Sixth embodiment)

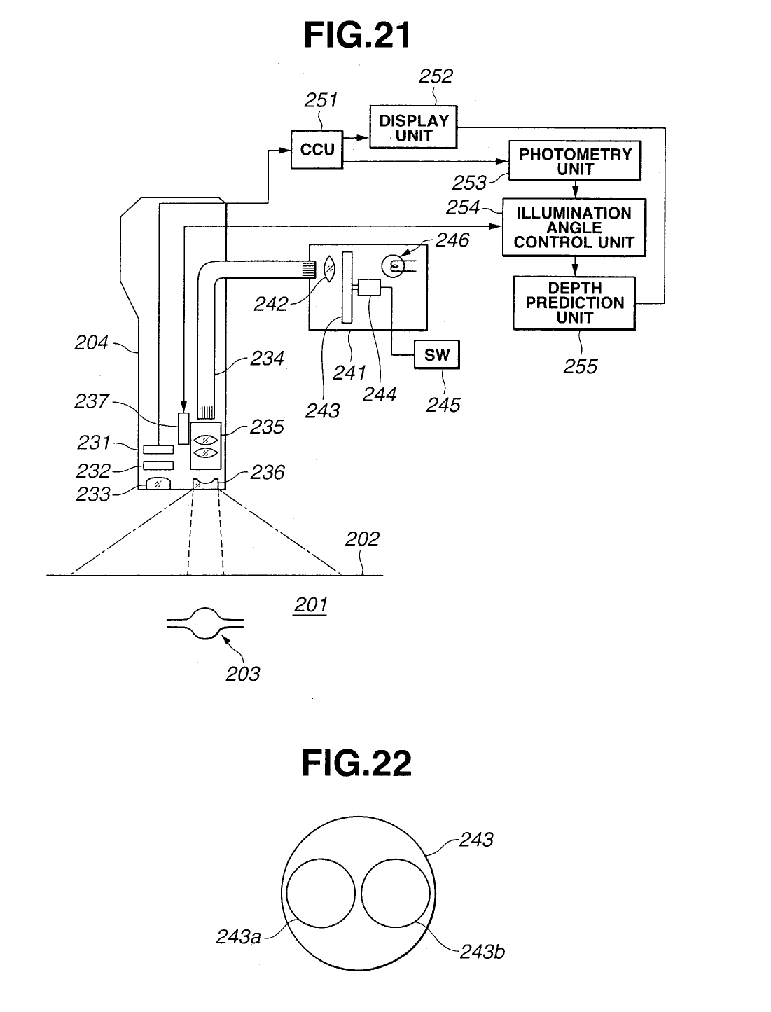

FIGS. 21 through 23 are diagrams for describing a sentinel lymph node detecting apparatus according to a sixth embodiment of the present invention. The apparatus according to the present embodiment is a sentinel lymph node detecting apparatus employing fluorescent dye.

FIG. 21 is a configuration diagram which illustrates a sentinel lymph node detecting apparatus according to the six embodiment. FIG. 22 is an explanation diagram which illustrates a configuration of a filter wheel.

In FIG. 21, reference numeral 201 denotes organic tissue, reference numeral 202 denotes a organic tissue surface, reference numeral 203 denotes a sentinel lymph node, and reference numeral 204 denotes an endoscope.

The endoscope 204 includes a CCD 231 which is a detector, an excitation light cut-off filter 232 which transmits white light and a part of infrared light, and cuts out excitation light, a condenser lens 233, an optical fiber 234, an illumination angle adjusting optical system 235, illumination lens 236, and an actuator 237.

The condenser lens 233 and the illumination lens 236 are disposed closely one to another. The optical fiber 234 is a light guiding means which guides light from a light source to the tip of the endoscope 204. The illumination angle adjusting optical system 235 is an optical system for adjusting the illumination angle of light which is cast from the output end at the tip of the optical fiber 234. The actuator 237 is an actuator for moving the illumination angle adjusting optical system 235.

Reference numeral 241 denotes a light source device. The light source device 241 includes a condenser lens 242, a filter wheel 243, a motor 244 for rotating the filter wheel 243, a switch 245 for driving the motor 244 in order to switch between observation with white light and fluorescent observation, and a light source lamp 246. The motor 244 receives signals from the switch 245, and rotates the filter wheel 243. Note that the light source lamp 246 is a light source which emits light containing infrared light and fluorescent excitation light.

The light from the lamp 246 of the light source device 241 is cast on the condenser lens 242 via one of two filters included in the filter wheel 243. The filter wheel 243 is a filter means having a configuration as shown in FIG. 22, which is a filter for illuminating in a manner wherein excitation light or white light is selected.

The filter wheel 243 is round in shape. The filter wheel 243 has an infrared cut-off filter 243a and an excitation light filter 243b. The infrared cut-off filter 243a cuts off infrared light contained in white light. On the other hand, the excitation light filter 243b transmits only excitation light which excites fluorescent dye such as ICG and generates fluorescence.

With the sentinel lymph node detecting apparatus, a user operates or control the switch 245 so as to drive the motor 244, so that the filter, which is to be inserted onto the light path from the lamp 246 to the condenser lens 242, can be switched either to the infrared cut-off filter 243a or excitation light filter 243b.

With the sentinel lymph node detecting apparatus, when disposing the infrared cut-off filter 243a on the light path, white light is output from the tip of the endoscope 204. Conversely, when disposing the excitation light filter 243b, excitation light for exciting fluorescent dye is output from the tip of the endoscope 204.

The light condensed by the condenser lens 242 is input to one end of the optical fiber 234, and is output from the output end, which is the other end on the tip side of the endoscope 204, of the optical fiber 234. The light output from the optical fiber 234 is cast on the illumination lens 236 via the illumination angle adjusting optical system 235. The illumination angle adjusting optical system 235 can be moved in the light path direction for the output light by the actuator 237. The actuator 237 is a piezoelectric type liner actuator, for example. With the sentinel lymph node detecting apparatus, the illumination angle of the light which is cast on the surface 202 of the organic tissue 201 from the illumination lens 236 can be enlarged or reduced by moving the illumination angle adjusting optical system 235 in the light axis direction of the output light, that is to say, the degree of condensation of light can be adjusted.

Reference numeral 251 denotes a camera control unit (which will be abbreviated to “CCD” hereafter), and reference numeral 252 denotes a display unit which is a monitor device. Reference numeral 253 denotes a photometry unit for measuring the brightness of a fluorescent image, reference numeral 254 denotes an illumination angle control unit, and reference numeral 255 denotes a depth prediction unit. The CCU 251 receives image signals from the CCD 231, and generates reflected-light images and fluorescent images. The display unit 252 displays endoscope images, and also displays the position information with regard to the depth-wise direction which will be described later. The illumination angle control unit 254 drives the actuator 237 and controls movement of the illumination angle adjustment optical system 235, so that the brightness of the fluorescent image is a predetermined constant value. The depth prediction unit 255 predicts the position of a sentinel lymph node in the depth-wise direction based upon the brightness of the fluorescent image.

The ICG emits fluorescence due to the excitation light cast on the surface 202 of the organic tissue 201. The fluorescence is cast on the CCD 231 via the condenser lens 233 and the excitation light cut-off filter 232. The image signals from the CCD 231 are input to the CCU 251, and are supplied to the display unit 252 as a two-dimensional image. Also, the image signals from the CCU 251 are output to the photometry unit 253. With the sentinel lymph node detecting apparatus, the illumination angle is controlled based upon photometry signals measured by the photometry unit 253, so the photometry signals are supplied to the illumination angle control unit 254.

The illumination angle control unit 254 controls the illumination angle by driving the actuator 237 so that the signal from the CCD 231 is equal to or greater than a predetermined value. The depth prediction unit 255 predicts the position, at which a sentinel lymph node is situated, from the surface 202, that is to say, the depth, based upon the output signals from the illumination angle control unit 254.

Next, operations of the sentinel lymph node detecting apparatus described above will be described.

In the event of using the endoscope 204 in ordinary observation with visible light, a surgeon operates the endoscope 204 so that the tip of the endoscope 204 approaches near the affected portion on the surface 202 of the organic tissue 201 while observing the affected portion within the body cavity of a patient. In this case, the surgeon operates the switch 245 so that the infrared cut-off filter 243a of the filter wheel 243 is inserted between the lamp 246 and the condenser lens 242, and light is cast on one end of the optical fiber 234, which is a light guiding means, via the infrared cut-off filter 243a.

The light passes through the illumination angle adjusting optical system 235, and is cast on the surface 202 of the organic tissue 201 from the illumination lens 236. The reflected light from the surface 202 is received by the CCD 231 via the condenser lens 233 and the excitation light cut-off filter 232. The CCD 231 outputs images of the surface 202 to the CCU 251 as two-dimensional image signals. The CCU 251 performs image processing for image signals from the CCD 231 so that the image signal can be displayed on the monitor device, and outputs to the display unit 252. Thus, the surgeon can observe the surface 202 of the organic tissue 201.

In the event of detecting sentinel lymph nodes, the surgeon locally injects ICG around the affected portion of a patient beforehand. Following a predetermined time period for the injected ICG migrating from the injected portion to lymphatic vessels, the surgeon operates the endoscope 204 so that the tip of the endoscope 204 approaches near the surface 202 of the organic tissue 201 while observing around the affected portion within the body cavity of the patient. Subsequently, the surgeon operates the switch 245 so that the excitation light filter 243b is inserted between the lamp 246 and the condenser lens 242, and light is cast on one end of the optical fiber 234 via the excitation light filter 243b. The excitation light is cast on the surface 202 of the organic tissue 201 from the illumination lens 236. In the event that there are lymph nodes containing ICG, the fluorescence from the excited ICG is received by the CCD 231 via the condenser lens 233 and the excitation light cut-off filter 232. The CCD 231 outputs the state of fluorescence to the CCU 251 as two-dimensional image signals. The CCU 251 performs image processing for the image signals from the CCD 231 so that the image signals can be displayed on a monitor device, and outputs to the display unit 252. Thus, the sentinel lymph node detecting apparatus can detect sentinel lymph nodes and positions thereof within the organic tissue 201.

In the event that fluorescence is not detected, or the detected quantity of the fluorescence is insufficient even if excitation light is cast from the illumination lens 236, with this sentinel lymph node detecting apparatus the illumination angle control unit 254 drives the actuator 237 based upon the photometry signals from the photometry unit 253 so that the illumination angle of the excitation light cast from the illumination lens is reduced. Conversely, in the event that the detected quantity of the fluorescence is too large, with this sentinel lymph node detecting apparatus the illumination angle control unit 254 drives the actuator 237 based upon the photometry signals from the photometry unit 253 so that the illumination angle of the excitation light cast from the illumination lens is increased.

The depth prediction unit 255 correlates the relationship between the output from the illumination angle control unit 254 and the corresponding illumination angle. Accordingly, the depth prediction unit 255 predicts the depth-wise position of the sentinel lymph node within the organic tissue 201 based upon the output from the illumination angle control unit 254.

The depth prediction unit 255 outputs signals for prediction results to the display unit 252 so that the depth-wise positions of the sentinel lymph nodes which are the prediction results are displayed on a monitor device for notifying the surgeon or the like.

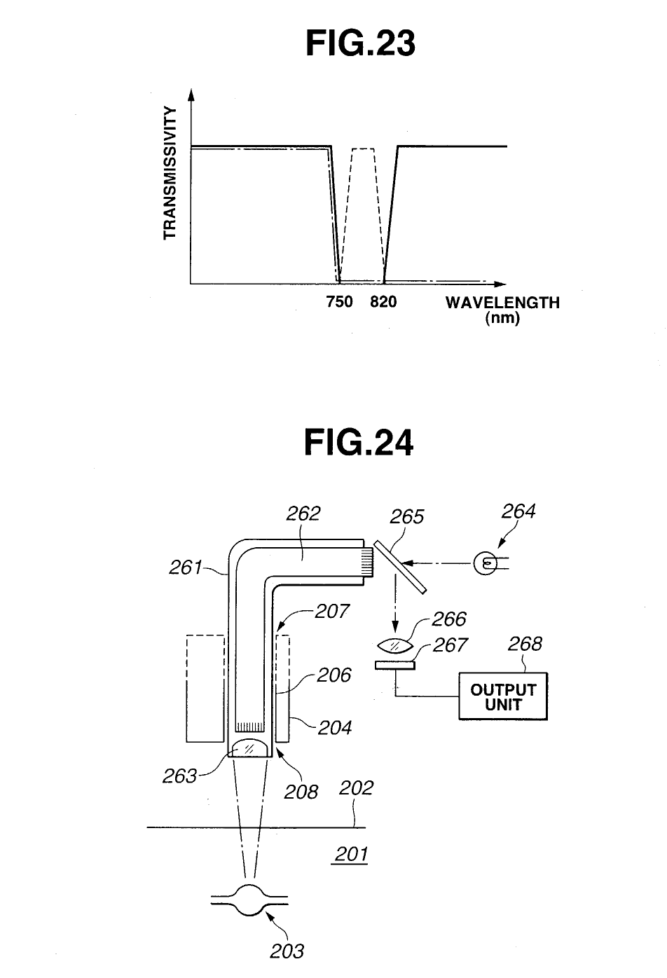

Now, the nature of light transmission with regard to each filter will be described. FIG. 23 is a light transmission characteristic diagram for each filter.

In FIG. 23, the single-dot broken line indicates the characteristic of the infrared cut-off filter 243a. The infrared cut-off filter 243a does not transmit light with a wavelength generally equal to or greater than 750 nm. Thus, in the event of observation as an ordinary endoscope with visible light, the fluorescence occurring due to the excited ICG is cut out from the light which has been filtered by the infrared cut-off filter 243a.

The broken line indicates the characteristics of the excitation light filter 243b. The excitation light filter 243b transmits only the light with a wavelength which is generally equal to or greater than 750 nm and generally equal to or less than 820 nm. Thus, in the event of detecting sentinel lymph nodes, the light which has been filtered by the excitation light filter 243b contains light with a wavelength, wherein the ICG is excited and emits fluorescence.

The solid line indicates the characteristics of the excitation light cut-off filter 232. The excitation light cut-off filter 232 does not transmit only the light with a wavelength which is generally equal to or greater than 750 nm and generally equal to or less than 820 nm.

Thus, in the event of observation with visible light, the CCD 231 can detect white light except for the excitation light. Conversely, in the event of detecting sentinel lymph nodes, the CCD 231 can detect the fluorescence excited due to excitation light, except for the excitation light.

As described above, with the present embodiment, a sentinel lymph node at a deep position can be detected, and the illumination angle can be altered by the illumination angle control unit, thereby detecting sentinel lymph nodes at various depth-wise positions.

Note that, while the above description has been made with regard to an arrangement wherein sentinel lymph nodes are detected by detecting fluorescence due to excited ICG, an arrangement may be made wherein sentinel lymph nodes are detected using the optoacoustic effect as described in the fifth embodiment.

That is to say, the sentinel lymph node detecting apparatus employs a pulse laser device as a light source lamp, and employs a piezoelectric element array instead of a CCD.

The pulse laser device casts a pulse laser beam on the area around the affected portion so as to generate ultrasonic signals due to the optoacoustic effect, the same as the fifth embodiment. The sentinel lymph node detecting apparatus then receives ultrasonic signals generated by dye by means of the piezoelectric element array, and generates two-dimensional images.

Thus, the present embodiment may be made as a detecting apparatus for sentinel lymph nodes using the optoacoustic effect.

(Seventh Embodiment)

Now, a seventh embodiment according to the present invention will be described.

FIG. 24 is a configuration diagram which illustrates a sentinel lymph node detecting apparatus according to the seventh embodiment. The apparatus according to the present embodiment is a sentinel lymph node detecting apparatus using fluorescent dye.

In FIG. 24, reference numeral 201 denotes organic tissue, reference numeral 202 denotes organic tissue surface, and reference numeral 203 denotes a sentinel lymph node. Reference numeral 204 denotes an endoscope which includes an imaging device (not shown) such as a CCD or the like, and outputs image signals for displaying images taken by the imaging device on a monitor device.

Reference numeral 261 denotes a probe for being inserted into the channel 206 of the endoscope 204 for a surgical instrument. The probe 261 includes an optical fiber 262 which is a light guide means inside. The probe 261 is inserted from the forceps opening 207, which is an inserting opening, provided to the operating unit of the ordinary endoscope 204, and can be protruded from the opening 208 at the tip of the endoscope 204. The probe 261 casts excitation light from the tip thereof so as to excite fluorescent dye, receives fluorescence from the dye, and guides the fluorescence to a detector which will be described later.

Specifically, a condenser lens 263 is provided to the tip of the probe 261. The optical fiber 262 guides the light from the light source lamp 264, and guides the light received via the condenser lens 263. The light source lamp 264 generates excitation light so that fluorescent dye such as ICG or the like generates fluorescence.

Reference numeral 265 denotes a dichroic mirror. Reference numeral 266 denotes a condenser lens, and reference numeral 267 denotes a detector for detecting fluorescence. Reference numeral 268 denotes an output unit.

The output unit 268 is an output device for receiving output signals, which are signals of the change in the fluorescence intensity, detected by the detector 267, and notifying a surgeon of the change in the fluorescence intensity by a light emitting diode (LED), buzzer, or the like. The condenser lens 263 is an optical system for condensing excitation light on a sentinel lymph node. The condenser lens 266 is an optical system for condensing the fluorescence from the dichroic mirror 265 on the detector 267. The dichroic mirror 265 is a mirror for passing the excitation light from the light source lamp 264, and reflecting the fluorescence from dye.

Now, operations of the sentinel lymph node detecting apparatus described above will be described.

A surgeon locally injects ICG around the affected portion of a patient beforehand. Following a predetermined time period for the injected dye to migrate from the injected portion to lymphatic vessels, the surgeon operates the endoscope 204 so that the tip of the probe 261 approaches the surface 202 of the organic tissue 201 while observing the affected portion within the body cavity of the patient. The surgeon operates a predetermined switch (not shown) so that the lamp 264 generates excitation light. The excitation light from the light source lamp 264 passes through the dichroic mirror 265, and enters the optical fiber 262 from the end of the optical fiber 262. The excitation light is output from the condenser lens 263, and is cast on the area around the affected tissue on the surface 202 of the organic tissue 201. The excitation light is near-infrared light in the wavelength range between 800 nm through 900 nm (nanometers), as described in the sixth embodiment. Upon ICG receiving such excitation light, the ICG emits fluorescence.

The fluorescence emitted by the ICG is condensed by the condenser lens 263, and is transmitted toward dichroic mirror 265 via the optical fiber 262. The dichroic mirror 265 transmits the excitation light, but reflects the fluorescence. Accordingly, the condenser lens 266 condenses the fluorescence toward the detector 267. The fluorescence is received by the detector 267 via the condenser lens 266. The detected signals are supplied to the output unit 268. The output unit 268 notifies the surgeon that sentinel lymph nodes are detected by turning on LEDs, or the like, in the event that the amplitude of the change in the detected signals with regard to time is sufficient as compared with a predetermined value.

Accordingly, the surgeon can recognize the presence of the ICG within the organic tissue 201 in front of the tip of the probe 261, that is to say, the presence of a sentinel lymph node.

With the present embodiment, incoming light is condensed by the condenser lens 263, so a sentinel lymph node at a further depth can be detected. Furthermore, a sentinel lymph node at a desired depth can be detected by changing the focal distance of the optical lens 263.

(Eighth Embodiment)

Now, an eighth embodiment according to the present invention will be described.

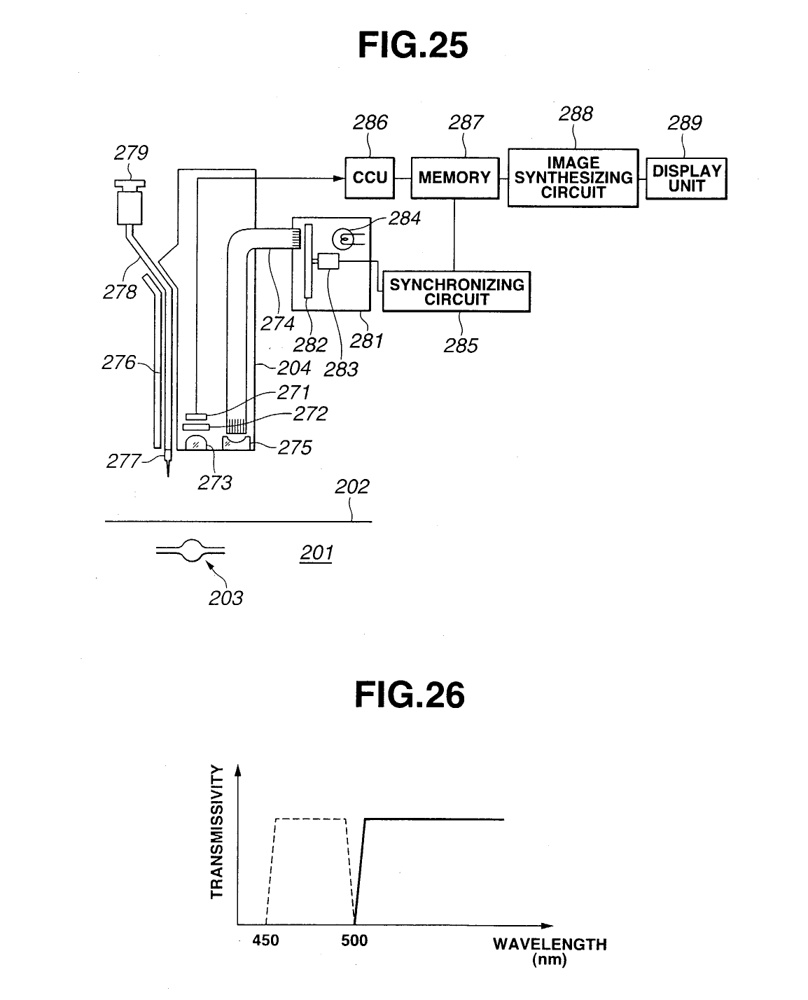

FIG. 25 is a configuration diagram which illustrates a sentinel lymph node detecting apparatus according to the eighth embodiment. The apparatus according to the present embodiment is a sentinel lymph node detecting apparatus employing a tracer, which emits fluorescence upon the tracer being combined with the affected portion.

In FIG. 25, reference numeral 201 denotes an organic tissue, reference numeral 202 denotes an organic tissue surface, reference numeral 203 denotes a sentinel lymph node, and reference numeral 204 denotes an endoscope.

The endoscope 204 includes a CCD 271 which is an imaging device, an excitation light cut-off filter 272 which cuts out excitation light and passes light with a wavelength greater than that of the excitation light, a condenser lens 273, an optical fiber 274, an illumination lens 275, and a channel 276 for a surgical instrument of the endoscope 204.

An injecting probe 278 having a needle 277 at the tip thereof can be inserted into the channel 276. The optical fiber 274 is a light guiding means, and guides the light from the light source to the tip of the endoscope. A surgeon can inject tracer fluid within an injector into a lower portion of mucous tissue, which is a lower portion of the affected tissue, from the tip of the needle 277 by pressing a syringe pump 279 of the injector. The tracer is a combination of antibodies which emit fluorescence when combined with the affected portion.

Reference numeral 281 denotes a light source device. The light source device 281 includes a filter wheel 282, a motor 283 for rotating the filter wheel 282, and a light source lamp 284. Note that the lamp 284 is a light source which emits light containing infrared light and fluorescence-excitation light. The motor 283 rotates the filter wheel 282, synchronized with a synchronizing circuit which will be described later.

The light from the lamp 284 of the light source device 281 is cast on one end of the optical fiber 274 via a filter of the filter wheel 282. The filter wheel 282 has the same configuration as the filter shown in FIG. 22 described above. The filter wheel 282 is a filter for switching excitation light and white light for lighting. The filter wheel 282 is round in shape. The filter wheel 282 has an infrared cut-off filter and an excitation light filter.

With the filter wheel 282, the motor 283 is driven according to signals from the synchronizing circuit 285, so that either or the other of the infrared cut-off filter and the excitation light filter is inserted on the light path between the lamp 284 and one end of the optical fiber 274, thereby enabling either of the infrared cut-off filter or the excitation light filter to be selected.

The light input to one end of the optical fiber 274 is output from the output end which is the other end of the optical fiber 274, which is a light guide, on the tip end side of the endoscope. The light output from the optical fiber 274 is cast on the illumination lens 275, and is cast on the surface 202 of the organic tissue 201 from the illumination lens 275.

The incident light diffuses from the surface 202 around the affected tissue into the organic tissue 201. The tracer which has been injected beforehand is a material which emits fluorescence upon receiving excitation light.

The light is cast on the CCD 271 via the condenser lens 273 and the excitation light cut-off filter 272.

Reference numeral 286 denotes a CCU, reference numeral 287 denotes memory, reference numeral 288 denotes an image synthesizing unit, and reference numeral 289 denotes a display unit which is a monitor device.

Image signals from the CCD 271 are input to the CCU 286, and image signals which are output signals are stored in the memory 287.

Specifically, when illuminating with white light, reflected-light images are stored in the memory 287, synchronously with signals from the synchronizing circuit 285. Conversely, when illuminating with excitation light, fluorescence images from the tracer which has been combined with the affected portion are stored in the memory 287, synchronously with signals from the synchronizing circuit 285.

The image signals stored in the memory 287 are synthesized in the image synthesizing unit 288, and the synthesized signals are output to the display unit 289 which is a monitor. That is to say, the image synthesizing unit 288 superimposes the fluorescence image on the reflected image, and the display unit 289 displays the synthesized image.

Now, the operations of the sentinel lymph node detecting apparatus described above will be described.

A surgeon locally injects a fluorescent antibody as a tracer around the affected portion beforehand. The fluorescent antibody is a material which emits fluorescence upon receiving excitation light in the state that the antibody is combined with the affected portion as a tracer. The fluorescent antibody is a monoclonal antibody, or a green fluorescence protein (which will be abbreviated to “GFP”), for example. The surgeon operates the endoscope 204 so that the needle 277 is inserted into the organic tissue 201 from the surface 202, and injects a fluorescent antibody into the organic tissue while observing the affected portion within the body cavity of the patient.

Following a predetermined time period for injected dye to migrate from the injected portion to lymphatic vessels, the surgeon operates the endoscope 204 so that the tip of the endoscope 204 approaches the surface 202 of the organic tissue 201 while observing the affected portion within the body cavity of the patient.

The surgeon operates a predetermined switch (not shown) so as to drive the synchronizing circuit 285.

The synchronizing circuit 285 drives the motor 283 so as to rotate the filter wheel 282 so that either of the infrared cut-off filter or the excitation light filter of the filter wheel 282 is inserted between the light source lamp 284 and the optical fiber 274 in an alternating manner. The light from the light source lamp 284 is filtered into excitation light or white light according to the rotation of the filter wheel 282, and the filtered light is input into the optical fiber 274. Upon white light being cast from the illumination lens 275, the CCD 271 supplies two-dimensional reflected-light images (signals) of the surface 202 of the organic tissue 201, received via the excitation light cut-off filter 272, to the CCU 286. Upon excitation light being cast from the illumination lens 275, the CCD 271 supplies two-dimensional fluorescence images from the fluorescent antibody to the CCU 286. The output from the synchronizing circuit 285 is a signal synchronous with the rotations of the filter wheel 282, and accordingly is used as a signal which indicates whether the light cast from the optical fiber 274 is white light or excitation light. Accordingly, the reflected-light image and fluorescence image are stored in the memory 287, respectively, according to the output signals from the synchronizing circuit 285. The two images stored in the memory 287 are supplied to the image synthesizing unit 288, and are synthesized. The image synthesizing unit 288 outputs video signals for displaying the synthesized image on a monitor, to the display unit 289.

Thus, the surgeon can observe the two-dimensional fluorescence image from GFP, which has been superimposed on the two-dimensional reflected-light image of the surface 202 of the organic tissue 201.

Now, the nature of light transmission with regard to each filter will be described. FIG. 26 is a light transmission characteristic diagram for each filter.

In FIG. 26, the broken indicates the characteristic of the excitation light filter of the filter wheel 282. The excitation light filter transmits only the light generally in the wavelength range between 450 nm through 500 nm (nanometers). Thus, when detecting sentinel lymph nodes, the light filtered by the excitation light filter contains light in the wavelength range which excites the GFP for generating fluorescence. The solid line indicates the characteristic of the excitation light cut-off filter 272. The excitation light cut-off filter 272 transmits only light with a wavelength generally greater than 500 nm. Accordingly, the CCD 271 can detect reflected light and fluorescence other than the excitation light.

As described above, with the eighth embodiment, sentinel lymph nodes can be detected using a material which emits fluorescence upon the material being combined with the affected portion as a tracer.

With the present invention, it is clear that a wide variety of embodiments may be made based upon the present invention without departing from the spirit and scope of the invention. The invention is not to be restricted by particular embodiments except as limited by the appended claims.

What is claimed is:

1. A sentinel lymph node detecting apparatus comprising:
fluctuating magnetic field generating means for vibrating ferrofluid, which has been accumulated in a sentinel lymph node around an affected portion beforehand, by the fluctuation of the magnetic field, so that the ferrofluid is heated;
endoscope imaging means for taking endoscope images around the affected portion;
temperature change imaging means for taking images of the change in temperature around the affected portion which has been heated due to the fluctuation of the magnetic field generated by the fluctuating magnetic field generating means; and
superimposing means for superimposing a temperature-change image obtained by the temperature change imaging means on an endoscope image obtained by the endoscope imaging means.

2. A sentinel lymph node detecting apparatus according to claim 1, wherein the endoscope imaging means is an optical endoscope for obtaining visible-light images around the affected portion.

3. A sentinel lymph node detecting apparatus according to claim 1, wherein the endoscope imaging means is an ultrasonic endoscope for obtaining ultrasonic tomographic images around the affected portion.

4. A sentinel lymph node detecting apparatus according to claim 1, wherein the temperature change imaging means is infrared imaging means for obtaining infrared images around the affected portion.

5. A sentinel lymph node detecting apparatus according to claim 1, wherein the temperature change imaging means is microwave imaging means for obtaining microwave image around the affected portion.

6. A sentinel lymph node detecting apparatus according to claim 1, wherein the temperature change imaging means is disposed on a probe which can be inserted into a surgical instrument inserting channel of the endoscope.

7. A sentinel lymph node detecting apparatus according to claim 2, wherein, with the optical endoscope, a catheter tube and an excision snare are inserted into the surgical instrument inserting channel, following which mucous tissue of the affected portion is removed with the excision snare, while sucking a tracer which has been accumulated in the affected portion due to injection thereof in order to identify the position of the sentinel lymph node.

8. A sentinel lymph node detecting apparatus according to claim 3, wherein the fluctuating magnetic field generating means is disposed on the tip of the inserting portion of the ultrasonic endoscope.

9. A sentinel lymph node detecting apparatus according to claim 3, wherein the ultrasonic endoscope acquires Doppler signals so as to obtain Doppler images as the temperature change imaging means, synchronous with the fluctuating magnetic field generating means.

10. A sentinel lymph node detecting apparatus according to claim 4, wherein the infrared images obtained by the infrared imaging means are computer color-enhanced images indicating the temperature distribution on the surface of organic tissue.

11. A sentinel lymph node detecting apparatus according to claim 4, wherein the infrared imaging means has a micro-bolometer array.

12. A sentinel lymph node detecting apparatus according to claim 5, wherein the infrared imaging means has a microwave antenna.

13. A sentinel lymph node detecting apparatus according to claim 6, wherein a tube channel into which a surgical instrument can be inserted is provided to the probe.

14. A sentinel lymph node detecting apparatus according to claim 6, wherein, with the probe, a suction cap is disposed on the tip thereof, and furthermore, a suction device for sucking tissue of a sentinel lymph node by air suctioning, which is connected to the tube channel, and a ring which is disposed within the cap, and marks the sucked tissue, are disposed.

15. A sentinel lymph node detecting apparatus according to claim 6, wherein, with the probe, a light guide for guiding infrared light is inserted and provided thereinto, and infrared imaging means is disposed at the base end thereof, as the temperature change imaging means.

16. A sentinel lymph node detecting apparatus according to claim 6, wherein the probe includes a suction needle for taking a tissue sample, which is inserted into the tube channel, an optical fiber which is inserted into the suction tube of the suction needle, a light source for supplying light to the optical fiber, a light intensity detector for detecting the light intensity of return light obtained by casting light from the light source on a tissue via the optical fiber, and notifying means for notifying that the tip of the suction needle has reached a sentinel lymph node based upon the change in the intensity of the return light detected by the light intensity detector.

17. A sentinel lymph node detecting apparatus according to claim 12, wherein a driving unit for rotationally or linearly driving the microwave antenna is provided to the base end of the probe.

18. A sentinel lymph node detecting apparatus according to claim 16, wherein, with the suction needle for taking a tissue sample, a two-forked unit is provided between the tip of the needle and the suction tube, and a fiber tube is provided to the other end of the forked unit on the branched side for the optical fiber being inserted.

19. A sentinel lymph node detecting method using a sentinel lymph node detecting apparatus, the apparatus comprising:
fluctuating magnetic field generating means for vibrating ferrofluid, which has been accumulated in a sentinel lymph node around an affected portion beforehand, by the fluctuation of the magnetic field, so that the ferrofluid is heated;
endoscope imaging means for taking endoscope images around the affected portion; and
temperature change imaging means for taking images of the change in temperature around the affected portion which has been heated due to the fluctuation of the magnetic field generated by the fluctuating magnetic field generating means;
wherein a temperature-change image obtained by the temperature change imaging means is superimposed on an endoscope image obtained by the endoscope imaging means, so as to identify the position of the sentinel lymph node.

20. A sentinel lymph node detecting apparatus comprising:
a pulse light source for casting pulse light for generating the change in ultrasonic signals with regard to time, occurring from dye due to the optoacoustic effect from absorption of light with a specific wavelength for the dye;
light guide means for guiding the pulse light around an affected portion into which the dye has been injected beforehand;
a detector which is disposed at a position close to the output end of the light guide means, and detects the ultrasonic signals; and
output means for outputting presence or absence of the dye, or the density of the dye, based upon output signals from the detector.

21. A sentinel lymph node detecting apparatus comprising:
a light source for exciting fluorescent dye which has been injected into a sentinel lymph node around an affected portion beforehand;
an endoscope having a light guide for guiding illumination light from the light source into the body cavity;
imaging means for observing fluorescence from the fluorescent dye, which is disposed on the tip of the endoscope; and
illumination angle adjusting means for adjusting the illumination angle of the illumination light into the body cavity, which is disposed between the light guide and the body cavity.

22. A sentinel lymph node detecting apparatus according to claim 21, wherein the illumination angle adjusting means is controlled by control means which adjusts the illumination angle, receiving the intensity signals of fluorescence received by the imaging means.

23. A sentinel lymph node detecting apparatus according to claim 21, wherein the fluorescent dye is an indocyanine green, a green fluorescence protein, or a monoclonal antibody.

24. A sentinel lymph node detecting apparatus according to claim 22, wherein the apparatus has depth prediction means for predicting the depth-wise position of the fluorescent dye emitting fluorescence based upon the illumination angle controlled by the control means.

25. A sentinel lymph node detecting apparatus comprising:
a light source which casts light for exciting a material, which has been injected around an affected portion, and emits fluorescence when the material being combined with the affected portion, and white light, as illumination light in an alternating manner;
an endoscope for outputting the illumination light from the light source via a light guide;
imaging means for observing fluorescence from the material, which is disposed on the tip of the endoscope;
recording means for recording reflected-light images and fluorescence images, synchronous with alternating casting of light from the light source; and
image synthesizing means for superimposing the fluorescence image on the reflected-light image, which are recorded on the recording means, and displaying the synthesized image.

26. A sentinel lymph node detecting method comprising:
a first step wherein a dye which absorbs light with a specific wavelength is injected around affected tissue beforehand;
a second step wherein pulse light which generates the change corresponding to time elapsing in ultrasonic signals occurring in the dye due to the optoacoustic effect from light with the wavelength, is cast on organic tissue to be observed around the affected tissue, via light guiding means; and
a third step wherein presence or absence of the dye, or the density of the dye, is output based upon output signals from a detector which is disposed at a position close to the output end of the light guide means, and detects the ultrasonic signals.
